SEQUENCE LISTING (8 sequences)

SEQ ID NO: 1 - DNA, 508 bp, Artificial Sequence
  human cytomegalovirus promoter such as the long CMV promoter, or cytomegalovirus (CMV) immediate-early enhancer - promoter
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagct

SEQ ID NO: 2 - DNA, 1189 bp, Artificial Sequence
  human EF1alpha promoter
cgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgag

SEQ ID NO: 3 - DNA, 516 bp, Artificial Sequence
  Human PGK promoter
ccacggggttggggttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcagggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccagg

SEQ ID NO: 4 - DNA, 409 bp, Artificial Sequence
  Spleen focus forming virus (SFFV) promoter
gtaacgccattttgcaaggcatggaaaaataccaaaccaagaatagagaagttcagatcaagggcgggtacatgaaaatagctaacgttgggccaaacaggatatctgcggtgagcagtttcggccccggcccggggccaagaacagatggtcaccgcagtttcggccccggcccgaggccaagaacagatggtccccagatatggcccaaccctcagcagtttcttaagacccatcagatgtttccaggctcccccaaggacctgaaatgaccctgcgccttatttgaattaaccaatcagcctgcttctcgcttctgttcgcgcgcttctgcttcccgagctctataaaagagctcacaacccctcactcggcgcgccagtcctccgacagactgagtcgcccgggg

SEQ ID NO: 5 - DNA, 4792 bp, Artificial Sequence
  SFFV- TBET-ETS1-TCF7 expression cassette
gtaacgccattttgcaaggcatggaaaaataccaaaccaagaatagagaagttcagatcaagggcgggtacatgaaaatagctaacgttgggccaaacaggatatctgcggtgagcagtttcggccccggcccggggccaagaacagatggtcaccgcagtttcggccccggcccgaggccaagaacagatggtccccagatatggcccaaccctcagcagtttcttaagacccatcagatgtttccaggctcccccaaggacctgaaatgaccctgcgccttatttgaattaaccaatcagcctgcttctcgcttctgttcgcgcgcttctgcttcccgagctctataaaagagctcacaacccctcactcggcgcgccagtcctccgacagactgagtcggccggtcgaatcaagcttatcgataccgtcgacggatccggatccgatgggcatcgtggagccgggttgcggagacatgctgacgggcaccgagccgatgccggggagcgacgagggccgggcgcctggcgccgacccgcagcaccgctacttctacccggagccgggcgcgcaggacgcggacgagcgtcgcgggggcggcagcctggggtctccctacccggggggcgccttggtgcccgccccgccgagccgcttccttggagcctacgcctacccgccgcgaccccaggcggccggcttccccggcgcgggcgagtccttcccgccgcccgcggacgccgagggctaccagccgggcgagggctacgccgccccggacccgcgcgccgggctctacccggggccgcgtgaggactacgcgctacccgcgggactggaggtgtcggggaaactgagggtcgcgctcaacaaccacctgttgtggtccaagtttaatcagcaccagacagagatgatcatcaccaagcagggacggcggatgttcccattcctgtcatttactgtggccgggctggagcccaccagccactacaggatgtttgtggacgtggtcttggtggaccagcaccactggcggtaccagagcggcaagtgggtgcagtgtggaaaggccgagggcagcatgccaggaaaccgcctgtacgtccacccggactcccccaacacaggagcgcactggatgcgccaggaagtttcatttgggaaactaaagctcacaaacaacaagggggcgtccaacaatgtgacccagatgattgtgctccagtccctccataagtaccagccccggctgcatatcgttgaggtgaacgacggagagccagaggcagcctgcaacgcttccaacacgcatatctttactttccaagaaacccagttcattgccgtgactgcctaccagaatgccgagattactcagctgaaaattgataataacccctttgccaaaggattccgggagaactttgagtccatgtacacatctgttgacaccagcatcccctccccgcctggacccaactgtcaattccttgggggagatcactactctcctctcctacccaaccagtatcctgttcccagccgcttctaccccgaccttcctggccaggcgaaggatgtggttccccaggcttactggctgggggccccccgggaccacagctatgaggctgagtttcgagcagtcagcatgaagcctgcattcttgccctctgcccctgggcccaccatgtcctactaccgaggccaggaggtcctggcacctggagctggctggcctgtggcaccccagtaccctcccaagatgggcccggccagctggttccgccctatgcggactctgcccatggaacccggccctggaggctcagagggacggggaccagaggaccagggtccccccttggtgtggactgagattgcccccatccggccggaatccagtgattcaggactgggcgaaggagactctaagaggaggcgcgtgtccccctatccttccagtggtgacagctcctcccctgctggggccccttctccttttgataaggaagctgaaggacagttttataactattttcccaacggcagcggcgccacaaacttctctctgctaaagcaagcaggtgatgttgaagaaaaccccgggcctatgagctactttgtggattctgctgggagcagccccgtcccttactcagcgcctcgtcctgcagtggtgaggcaaggacctagcaacacttatgaagatcctcgaatgaactgtggtttccagtccaattatcaccagcaaagaccttgctaccccttttgggatgagatggcaactcaggaagttcctactggtcttgaacactgtgtctcagatatggaatgtgcagatgtcccactattaactccaagcagcaaagaaatgatgtctcaagcattaaaagctactttcagtggtttcactaaagaacagcaacgactggggatcccaaaagacccccggcagtggacagaaacccatgttcgggactgggtgatgtgggctgtgaatgaattcagcctgaaaggtgtagacttccagaagttctgtatgaatggagcagccctctgcgccctgggtaaagactgctttctcgagctggccccagactttgttggggacatcttatgggaacatctagagatcctgcagaaagaggatgtgaaaccatatcaagttaatggagtcaacccagcctatccagaatcccgctatacctcggattacttcattagctatggtattgagcatgcccagtgtgttccaccatcggagttctcagagcccagcttcatcacagagtcctatcagacgctccatcccatcagctcggaagagctcctctccctcaagtatgagaatgactacccctcggtcattctccgagaccctctccagacagacaccttgcagaatgactactttgctatcaaacaagaagtcgtcaccccagacaacatgtgcatggggaggaccagtcgtggtaaactcgggggccaggactcttttgaaagcatagagagctacgatagttgtgatcgcctcacccagtcctggagcagccagtcatctttcaacagcctgcagcgtgttccctcctatgacagcttcgactcagaggactatccggctgccctgcccaaccacaagcccaagggcaccttcaaggactatgtgcgggaccgtgctgacctcaataaggacaagcctgtcattcctgctgctgccctagctggctacacaggcagtggaccaatccagctatggcagtttcttctggaattactcactgataaatcctgtcagtcttttatcagctggacaggagatggctgggaattcaaactttctgacccagatgaggtggccaggagatggggaaagaggaaaaacaaacctaagatgaattatgagaaactgagccgtggcctacgctactattacgacaaaaacatcatccacaagacagcggggaaacgctacgtgtaccgctttgtgtgtgacctgcagagcctgctggggtacacccctgaggagctgcacgccatgctggacgtcaagccagatgccgacgagggctccggcgagggcaggggaagtcttctaacatgcggggacgtggaggaaaatcccggcccaatgccgcagctggactccggcgggggcggcgcgggcggcggcgacgacctcggcgcgccggacgagctgctggccttccaggatgaaggcgaggagcaggacgacaagagccgcgacagcgccgccggtcccgagcgcgacctggccgagctcaagtcgtcgctcgtgaacgagtccgagggcgcggccggcggcgcagggatcccgggggtcccgggggccggcgccggggcccgcggcgaggccgaggctctcgggcgggaacacgctgcgcagagactcttcccggacaaacttccagagcccctggaggacggcctgaaggccccggagtgcaccagcggcatgtacaaagagaccgtctactccgccttcaatctgctcatgcattacccacccccctcgggagcagggcagcacccccagccgcagcccccgctgcacaaggccaatcagcccccccacggtgtcccccaactctctctctacgaacatttcaacagcccacatcccacccctgcacctgcggacatcagccagaagcaagttcacaggcctctgcagacccctgacctctctggcttctactccctgacctcaggcagcatggggcagctcccccacactgtgagctggttcacccacccatccttgatgctaggttctggtgtacctggtcacccagcagccatcccccacccggccattgtgcccccctcagggaagcaggagctgcagcccttcgaccgcaacctgaagacacaagcagagtccaaggcagagaaggaggccaagaagccaaccatcaagaagcccctcaatgccttcatgctgtacatgaaggagatgagagccaaggtcattgcagagtgcacacttaaggagagcgctgccatcaaccagatcctgggccgcaggtggcacgcgctgtcgcgagaagagcaggccaagtactatgagctggcccgcaaggagaggcagctgcacatgcagctatacccaggctggtcagcgcgggacaactacgggaagaagaagaggcggtcgagggaaaagcaccaagaatccaccacaggaggaaaaagaaatgcattcggtacttacccggagaaggccgctgccccagccccgttccttccgatgacagtgctctag

SEQ ID NO: 6 - DNA, 4802 bp, Artificial Sequence
  Fuw-TetO- TBET-ETS1-TCF7  expression cassette
tccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagctcggtacccgggtcgagtaggcgtgtacggtgggaggcctatataagcagagctcgtttagtgaaccgtcagatcgcctggagacgccatccacgctgttttgacctccatagaagacaccgggaccgatccagcctccgcggccccgaattcaccggttctagagctagcatgggcatcgtggagccgggttgcggagacatgctgacgggcaccgagccgatgccggggagcgacgagggccgggcgcctggcgccgacccgcagcaccgctacttctacccggagccgggcgcgcaggacgcggacgagcgtcgcgggggcggcagcctggggtctccctacccggggggcgccttggtgcccgccccgccgagccgcttccttggagcctacgcctacccgccgcgaccccaggcggccggcttccccggcgcgggcgagtccttcccgccgcccgcggacgccgagggctaccagccgggcgagggctacgccgccccggacccgcgcgccgggctctacccggggccgcgtgaggactacgcgctacccgcgggactggaggtgtcggggaaactgagggtcgcgctcaacaaccacctgttgtggtccaagtttaatcagcaccagacagagatgatcatcaccaagcagggacggcggatgttcccattcctgtcatttactgtggccgggctggagcccaccagccactacaggatgtttgtggacgtggtcttggtggaccagcaccactggcggtaccagagcggcaagtgggtgcagtgtggaaaggccgagggcagcatgccaggaaaccgcctgtacgtccacccggactcccccaacacaggagcgcactggatgcgccaggaagtttcatttgggaaactaaagctcacaaacaacaagggggcgtccaacaatgtgacccagatgattgtgctccagtccctccataagtaccagccccggctgcatatcgttgaggtgaacgacggagagccagaggcagcctgcaacgcttccaacacgcatatctttactttccaagaaacccagttcattgccgtgactgcctaccagaatgccgagattactcagctgaaaattgataataacccctttgccaaaggattccgggagaactttgagtccatgtacacatctgttgacaccagcatcccctccccgcctggacccaactgtcaattccttgggggagatcactactctcctctcctacccaaccagtatcctgttcccagccgcttctaccccgaccttcctggccaggcgaaggatgtggttccccaggcttactggctgggggccccccgggaccacagctatgaggctgagtttcgagcagtcagcatgaagcctgcattcttgccctctgcccctgggcccaccatgtcctactaccgaggccaggaggtcctggcacctggagctggctggcctgtggcaccccagtaccctcccaagatgggcccggccagctggttccgccctatgcggactctgcccatggaacccggccctggaggctcagagggacggggaccagaggaccagggtccccccttggtgtggactgagattgcccccatccggccggaatccagtgattcaggactgggcgaaggagactctaagaggaggcgcgtgtccccctatccttccagtggtgacagctcctcccctgctggggccccttctccttttgataaggaagctgaaggacagttttataactattttcccaacggcagcggcgccacaaacttctctctgctaaagcaagcaggtgatgttgaagaaaaccccgggcctatgagctactttgtggattctgctgggagcagccccgtcccttactcagcgcctcgtcctgcagtggtgaggcaaggacctagcaacacttatgaagatcctcgaatgaactgtggtttccagtccaattatcaccagcaaagaccttgctaccccttttgggatgagatggcaactcaggaagttcctactggtcttgaacactgtgtctcagatatggaatgtgcagatgtcccactattaactccaagcagcaaagaaatgatgtctcaagcattaaaagctactttcagtggtttcactaaagaacagcaacgactggggatcccaaaagacccccggcagtggacagaaacccatgttcgggactgggtgatgtgggctgtgaatgaattcagcctgaaaggtgtagacttccagaagttctgtatgaatggagcagccctctgcgccctgggtaaagactgctttctcgagctggccccagactttgttggggacatcttatgggaacatctagagatcctgcagaaagaggatgtgaaaccatatcaagttaatggagtcaacccagcctatccagaatcccgctatacctcggattacttcattagctatggtattgagcatgcccagtgtgttccaccatcggagttctcagagcccagcttcatcacagagtcctatcagacgctccatcccatcagctcggaagagctcctctccctcaagtatgagaatgactacccctcggtcattctccgagaccctctccagacagacaccttgcagaatgactactttgctatcaaacaagaagtcgtcaccccagacaacatgtgcatggggaggaccagtcgtggtaaactcgggggccaggactcttttgaaagcatagagagctacgatagttgtgatcgcctcacccagtcctggagcagccagtcatctttcaacagcctgcagcgtgttccctcctatgacagcttcgactcagaggactatccggctgccctgcccaaccacaagcccaagggcaccttcaaggactatgtgcgggaccgtgctgacctcaataaggacaagcctgtcattcctgctgctgccctagctggctacacaggcagtggaccaatccagctatggcagtttcttctggaattactcactgataaatcctgtcagtcttttatcagctggacaggagatggctgggaattcaaactttctgacccagatgaggtggccaggagatggggaaagaggaaaaacaaacctaagatgaattatgagaaactgagccgtggcctacgctactattacgacaaaaacatcatccacaagacagcggggaaacgctacgtgtaccgctttgtgtgtgacctgcagagcctgctggggtacacccctgaggagctgcacgccatgctggacgtcaagccagatgccgacgagggctccggcgagggcaggggaagtcttctaacatgcggggacgtggaggaaaatcccggcccaatgccgcagctggactccggcgggggcggcgcgggcggcggcgacgacctcggcgcgccggacgagctgctggccttccaggatgaaggcgaggagcaggacgacaagagccgcgacagcgccgccggtcccgagcgcgacctggccgagctcaagtcgtcgctcgtgaacgagtccgagggcgcggccggcggcgcagggatcccgggggtcccgggggccggcgccggggcccgcggcgaggccgaggctctcgggcgggaacacgctgcgcagagactcttcccggacaaacttccagagcccctggaggacggcctgaaggccccggagtgcaccagcggcatgtacaaagagaccgtctactccgccttcaatctgctcatgcattacccacccccctcgggagcagggcagcacccccagccgcagcccccgctgcacaaggccaatcagcccccccacggtgtcccccaactctctctctacgaacatttcaacagcccacatcccacccctgcacctgcggacatcagccagaagcaagttcacaggcctctgcagacccctgacctctctggcttctactccctgacctcaggcagcatggggcagctcccccacactgtgagctggttcacccacccatccttgatgctaggttctggtgtacctggtcacccagcagccatcccccacccggccattgtgcccccctcagggaagcaggagctgcagcccttcgaccgcaacctgaagacacaagcagagtccaaggcagagaaggaggccaagaagccaaccatcaagaagcccctcaatgccttcatgctgtacatgaaggagatgagagccaaggtcattgcagagtgcacacttaaggagagcgctgccatcaaccagatcctgggccgcaggtggcacgcgctgtcgcgagaagagcaggccaagtactatgagctggcccgcaaggagaggcagctgcacatgcagctatacccaggctggtcagcgcgggacaactacgggaagaagaagaggcggtcgagggaaaagcaccaagaatccaccacaggaggaaaaagaaatgcattcggtacttacccggagaaggccgctgccccagccccgttccttccgatgacagtgctctag

SEQ ID NO: 7 - RNA, 4344 nt, Artificial Sequence
  exemplary mRNA encoding TBET-ETS1-TCF7
augggcaucguggagccggguugcggagacaugcugacgggcaccgagccgaugccggggagcgacgagggccgggcgccuggcgccgacccgcagcaccgcuacuucuacccggagccgggcgcgcaggacgcggacgagcgucgcgggggcggcagccuggggucucccuacccggggggcgccuuggugcccgccccgccgagccgcuuccuuggagccuacgccuacccgccgcgaccccaggcggccggcuuccccggcgcgggcgaguccuucccgccgcccgcggacgccgagggcuaccagccgggcgagggcuacgccgccccggacccgcgcgccgggcucuacccggggccgcgugaggacuacgcgcuacccgcgggacuggaggugucggggaaacugagggucgcgcucaacaaccaccuguugugguccaaguuuaaucagcaccagacagagaugaucaucaccaagcagggacggcggauguucccauuccugucauuuacuguggccgggcuggagcccaccagccacuacaggauguuuguggacguggucuugguggaccagcaccacuggcgguaccagagcggcaagugggugcaguguggaaaggccgagggcagcaugccaggaaaccgccuguacguccacccggacucccccaacacaggagcgcacuggaugcgccaggaaguuucauuugggaaacuaaagcucacaaacaacaagggggcguccaacaaugugacccagaugauugugcuccagucccuccauaaguaccagccccggcugcauaucguugaggugaacgacggagagccagaggcagccugcaacgcuuccaacacgcauaucuuuacuuuccaagaaacccaguucauugccgugacugccuaccagaaugccgagauuacucagcugaaaauugauaauaaccccuuugccaaaggauuccgggagaacuuugaguccauguacacaucuguugacaccagcauccccuccccgccuggacccaacugucaauuccuugggggagaucacuacucuccucuccuacccaaccaguauccuguucccagccgcuucuaccccgaccuuccuggccaggcgaaggaugugguuccccaggcuuacuggcugggggccccccgggaccacagcuaugaggcugaguuucgagcagucagcaugaagccugcauucuugcccucugccccugggcccaccauguccuacuaccgaggccaggagguccuggcaccuggagcuggcuggccuguggcaccccaguacccucccaagaugggcccggccagcugguuccgcccuaugcggacucugcccauggaacccggcccuggaggcucagagggacggggaccagaggaccaggguccccccuugguguggacugagauugcccccauccggccggaauccagugauucaggacugggcgaaggagacucuaagaggaggcgcgugucccccuauccuuccaguggugacagcuccuccccugcuggggccccuucuccuuuugauaaggaagcugaaggacaguuuuauaacuauuuucccaacggcagcggcgccacaaacuucucucugcuaaagcaagcaggugauguugaagaaaaccccgggccuaugagcuacuuuguggauucugcugggagcagccccgucccuuacucagcgccucguccugcaguggugaggcaaggaccuagcaacacuuaugaagauccucgaaugaacugugguuuccaguccaauuaucaccagcaaagaccuugcuaccccuuuugggaugagauggcaacucaggaaguuccuacuggucuugaacacugugucucagauauggaaugugcagaugucccacuauuaacuccaagcagcaaagaaaugaugucucaagcauuaaaagcuacuuucagugguuucacuaaagaacagcaacgacuggggaucccaaaagacccccggcaguggacagaaacccauguucgggacugggugaugugggcugugaaugaauucagccugaaagguguagacuuccagaaguucuguaugaauggagcagcccucugcgcccuggguaaagacugcuuucucgagcuggccccagacuuuguuggggacaucuuaugggaacaucuagagauccugcagaaagaggaugugaaaccauaucaaguuaauggagucaacccagccuauccagaaucccgcuauaccucggauuacuucauuagcuaugguauugagcaugcccaguguguuccaccaucggaguucucagagcccagcuucaucacagaguccuaucagacgcuccaucccaucagcucggaagagcuccucucccucaaguaugagaaugacuaccccucggucauucuccgagacccucuccagacagacaccuugcagaaugacuacuuugcuaucaaacaagaagucgucaccccagacaacaugugcauggggaggaccagucgugguaaacucgggggccaggacucuuuugaaagcauagagagcuacgauaguugugaucgccucacccaguccuggagcagccagucaucuuucaacagccugcagcguguucccuccuaugacagcuucgacucagaggacuauccggcugcccugcccaaccacaagcccaagggcaccuucaaggacuaugugcgggaccgugcugaccucaauaaggacaagccugucauuccugcugcugcccuagcuggcuacacaggcaguggaccaauccagcuauggcaguuucuucuggaauuacucacugauaaauccugucagucuuuuaucagcuggacaggagauggcugggaauucaaacuuucugacccagaugagguggccaggagauggggaaagaggaaaaacaaaccuaagaugaauuaugagaaacugagccguggccuacgcuacuauuacgacaaaaacaucauccacaagacagcggggaaacgcuacguguaccgcuuugugugugaccugcagagccugcugggguacaccccugaggagcugcacgccaugcuggacgucaagccagaugccgacgagggcuccggcgagggcaggggaagucuucuaacaugcggggacguggaggaaaaucccggcccaaugccgcagcuggacuccggcgggggcggcgcgggcggcggcgacgaccucggcgcgccggacgagcugcuggccuuccaggaugaaggcgaggagcaggacgacaagagccgcgacagcgccgccggucccgagcgcgaccuggccgagcucaagucgucgcucgugaacgaguccgagggcgcggccggcggcgcagggaucccgggggucccgggggccggcgccggggcccgcggcgaggccgaggcucucgggcgggaacacgcugcgcagagacucuucccggacaaacuuccagagccccuggaggacggccugaaggccccggagugcaccagcggcauguacaaagagaccgucuacuccgccuucaaucugcucaugcauuacccaccccccucgggagcagggcagcacccccagccgcagcccccgcugcacaaggccaaucagcccccccacggugucccccaacucucucucuacgaacauuucaacagcccacaucccaccccugcaccugcggacaucagccagaagcaaguucacaggccucugcagaccccugaccucucuggcuucuacucccugaccucaggcagcauggggcagcucccccacacugugagcugguucacccacccauccuugaugcuagguucugguguaccuggucacccagcagccaucccccacccggccauugugccccccucagggaagcaggagcugcagcccuucgaccgcaaccugaagacacaagcagaguccaaggcagagaaggaggccaagaagccaaccaucaagaagccccucaaugccuucaugcuguacaugaaggagaugagagccaaggucauugcagagugcacacuuaaggagagcgcugccaucaaccagauccugggccgcagguggcacgcgcugucgcgagaagagcaggccaaguacuaugagcuggcccgcaaggagaggcagcugcacaugcagcuauacccaggcuggucagcgcgggacaacuacgggaagaagaagaggcggucgagggaaaagcaccaagaauccaccacaggaggaaaaagaaaugcauucgguacuuacccggagaaggccgcugccccagccccguuccuuccgaugacagugcucuag

SEQ ID NO: 8 - DNA, 8477 bp, Artificial Sequence
  FUW-tetO-MCS - addgene plasmid 84008
cgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggttatcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagctcggtacccgggtcgaggtaggcgtgtacggtgggaggcctatataagcagagctcgtttagtgaaccgtcagatcgcctggagacgccatccacgctgttttgacctccatagaagacaccgggaccgatccagcctccgcggccccgaattacaggctagctatcaggatcctacgaggcgcgccagatagaattctcttgggcctcaagggccatgagttaaccagttctagatgtgactcatgtcaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgacctcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaacgaagacaagatatccttgatctgtggatctaccacacacaaggctacttccctgattggcagaactacacaccagggccagggatcagatatccactgacctttggatggtgctacaagctagtaccagttgagcaagagaaggtagaagaagccaatgaaggagagaacacccgcttgttacaccctgtgagcctgcatgggatggatgacccggagagagaagtattagagtggaggtttgacagccgcctagcatttcatcacatggcccgagagctgcatccggactgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcg

(19) United States
(12) Patent Application Publication
(10) Pub. No.: US 2024/0166996 A1
(43) Pub. Date: May 23, 2024

(54) GENERATION OF T-CELLS BY DIRECT REPROGRAMMING FROM FIBROBLASTS AND MSC

(71) Applicant: AUTOLUS LIMITED, London (GB)

(72) Inventors: Carlos Fillipe Ribeiro Lemos Pereira, Lund (SE); Martin Pulé, London (GB)

(21) Appl. No.: 18/279,568

(22) PCT Filed: Mar. 4, 2022
(86) PCT No.: PCT/GB2022/050574

§ 371 Date: Aug. 30, 2023

(30) Foreign Application Priority Data
Mar. 5, 2021 (GB) 2103101.8

(51) Int. Cl. C12N 5/0783; C12N 15/87

(52) U.S. Cl. CPC C12N 5/0636; C12N 15/87; C12N 2501/2307; C12N 2501/2315; C12N 2501/60; C12N 2506/1353

(57) ABSTRACT

The invention relates to a method comprising (a) providing a vertebrate somatic cell, (b) inducing expression in said cell of at least three transcription factors, wherein said transcription factors are: i. Tbet (Tbx21), ii. Tcf7, and iii. Ets1; (c) incubating said cell to allow transdifferentiation. Suitably said cell is transdifferentiated to an immune effector cell. The invention also relates to cells, nucleic acids, pharmaceutical compositions, and methods of treatment.

### FIELD OF THE INVENTION

The invention relates to methods for transdifferentiation of somatic cells, in particular expandable somatic cells, to immune effector cells.

### BACKGROUND TO THE INVENTION

Immune effector CAR T-cells (sometimes referred to simply as “CAR T-cells”) are typically generated from autologous peripheral mononuclear cells. This is limiting because an individual production for each patient must be undertaken. Known off-the-shelf approaches use normal donor pheresis. Relatively undifferentiated immune effector cells are preferred. However, since expansion of immune effector cells is coupled with differentiation, it is not possible to generate large numbers of products from a single donor, which is a problem with this approach. A further drawback is that economies of scale cannot be applied.

Alternative approaches have been described which utilise induced progenitor cells (iPS) to develop immune effector CAR cells. This approach involves first de-differentiating a somatic cell (e.g. a skin fibroblast) into an iPS, and then differentiating the iPS into an immune effector cell. If desired, CAR T-cell genes can be introduced early in the process. Given that copious proliferation occurs throughout differentiation and the process can be halted at exactly the right differentiation step, effector CAR cells of the correct differentiation state could be made, and made in large numbers. However, this process suffers from drawbacks of being highly complex, and requiring highly skilled personnel, which factors make it time consuming and laborious. These are further problems in the art.

The present invention seeks to overcome problem(s) associated with the art.

### SUMMARY OF THE INVENTION

The inventors have been researching transdifferentiation. This is a process that involves reprogramming of differentiated cells without having to revert the cells back to a pluripotent stage. An advantage of this approach is that the final cell types of these reprogramming processes, progenitors and mature cells, may be directly used in applications such as cell replacement therapies.

The inventors describe an approach whereby somatic cells which are capable of in vitro expansion are directly transdifferentiated into immune effector cells. Thus, the invention teaches starting from a differentiated cell and directly transdifferentiating it into an immune effector cell. This has the advantage of avoiding use of problematic iPSCs. This has the further advantage of being simpler and less laborious than known iPSC or ESC based approaches, saving both time and cost.

The invention is based on these surprising findings.

Thus in one aspect the invention provides a method comprising
(a) providing a vertebrate somatic cell,
(b) inducing expression in said cell of at least three transcription factors, wherein said transcription factors are:
   i. Tbet (Tbx21),
   ii. Tcf7, and
   iii. Ets1;
(c) incubating said cell to allow transdifferentiation.

In another aspect the invention provides a method comprising
(a) providing a vertebrate somatic cell,
(b) inducing expression in said cell of at least three transcription factors, wherein said transcription factors are:
   i. Tbet (Tbx21),
   ii. Tcf7, and
   iii. Ets1;
(c) incubating said cell.

In another aspect the invention provides a method comprising
(a) providing a vertebrate somatic cell,
(b) inducing expression in said cell of at least three transcription factors, wherein said transcription factors are:
   i. Tbet (Tbx21),
   ii. Tcf7, and
   iii. Ets1.

In another aspect the invention provides a method as described above, further comprising inducing expression in said cell of one or more transcription factors selected from the group consisting of: (Bcl11b), (Satb1), (Gata3), (Satb1 and Rorc), (Bcl11b and Lef1), (Bcl11b and Ikzf1), (Gata3 and Ikzf1) and (Gata3 and Satb1 and Bcl11b).

In another aspect the invention provides a method comprising
(a) providing a vertebrate somatic cell,
(bi) inducing expression in said cell of at least three transcription factors, wherein said transcription factors are:
   Tbet (Tbx21), Tcf7, and Ets1;
(bii) further comprising inducing expression in said cell of transcription factor Bcl11b;
(c) incubating said cell to allow transdifferentiation.

In another aspect the invention provides a method comprising
(a) providing a vertebrate somatic cell,
(bi) inducing expression in said cell of at least three transcription factors, wherein said transcription factors are:
   Tbet (Tbx21), Tcf7, and Ets1;
(bii) further comprising inducing expression in said cell of transcription factor Satb1;
(c) incubating said cell to allow transdifferentiation.

In another aspect the invention provides a method comprising
(a) providing a vertebrate somatic cell,
(bi) inducing expression in said cell of at least three transcription factors, wherein said transcription factors are:
   Tbet (Tbx21), Tcf7, and Ets1;
(bii) further comprising inducing expression in said cell of transcription factor Gata3;
(c) incubating said cell to allow transdifferentiation.

In another aspect the invention provides a method comprising
(a) providing a vertebrate somatic cell,
(bi) inducing expression in said cell of at least three transcription factors, wherein said transcription factors are:
   Tbet (Tbx21), Tcf7, and Ets1;
(bii) further comprising inducing expression in said cell of transcription factors Satb1 and Rorc;
(c) incubating said cell to allow transdifferentiation.

In another aspect the invention provides a method comprising
(a) providing a vertebrate somatic cell,
(bi) inducing expression in said cell of at least three transcription factors, wherein said transcription factors are:
   Tbet (Tbx21), Tcf7, and Ets1;
(bii) further comprising inducing expression in said cell of transcription factors Bcl11b and Lef1;
(c) incubating said cell to allow transdifferentiation.

In another aspect the invention provides a method comprising
(a) providing a vertebrate somatic cell,
(bi) inducing expression in said cell of at least three transcription factors, wherein said transcription factors are:
   Tbet (Tbx21), Tcf7, and Ets1;
(bii) further comprising inducing expression in said cell of transcription factors Bcl11b and Ikzf1;
(c) incubating said cell to allow transdifferentiation.

In another aspect the invention provides a method comprising
(a) providing a vertebrate somatic cell,
(bi) inducing expression in said cell of at least three transcription factors, wherein said transcription factors are:
   Tbet (Tbx21), Tcf7, and Ets1;
(bii) further comprising inducing expression in said cell of transcription factors Gata3 and Ikzf1;
(c) incubating said cell to allow transdifferentiation.

In another aspect the invention provides a method comprising
(a) providing a vertebrate somatic cell,
(bi) inducing expression in said cell of at least three transcription factors, wherein said transcription factors are:
   Tbet (Tbx21), Tcf7, and Ets1;
(bii) further comprising inducing expression in said cell of transcription factors Gata3 and Satb1 and Bcl11b;
(c) incubating said cell to allow transdifferentiation.

In another embodiment the invention relates to a method as described above wherein step (b) comprises inducing expression in said cell of each of the transcription factors:
i. Tbet (Tbx21)
ii. Tcf7
iii. Ets1
iv. Bcl11b
v. Satb1
vi. Gata3
vii. Lef1
viii. Rorc; and
ix. Ikzf1.

In another embodiment the invention relates to a method as described above further comprising inducing expression in said cell of transcription factors
x. Nfatc3, and
xi. Nr4a3.

In another embodiment the invention relates to a method as described above further comprising inducing expression in said cell of transcription factors
xii. Stat4
xiii. Tox
xiv. Zbtb16
xv. Nfil3
xvi. Znf683
xvii. Tcf12.

In another embodiment the invention relates to a method as described above wherein said cell is transdifferentiated to an immune effector cell.

In another embodiment the invention relates to a method as described above wherein said cell is transdifferentiated to an alpha/beta T cell, or a gamma/delta T cell.

In another embodiment the invention relates to a method as described above wherein said cell is transdifferentiated to a CD8+ T cell.

In another embodiment the invention relates to a method as described above wherein said cell is transdifferentiated to a CD4+ T cell.

In another embodiment the invention relates to a method as described above wherein the vertebrate somatic cell is selected from the group consisting of: a mesenchymal stem cell (MSC), a skin fibroblast, an endothelial cell, a keratinocyte, and a hepatocyte.

In another embodiment the invention relates to a method as described above wherein said vertebrate somatic cell is selected from the group consisting of: a mesenchymal stem cell and a skin fibroblast.

In another embodiment the invention relates to a method as described above wherein the vertebrate somatic cell is a mesenchymal stem cell.

In another embodiment the invention relates to a method as described above wherein the vertebrate somatic cell is a skin fibroblast.

Suitably the vertebrate somatic cell is a human mesenchymal stem cell (hMSC). Suitably said hMSC is derived from bone marrow.

In another embodiment the invention relates to a method as described above wherein the immune effector cell of (c) is a cell which expresses one or more gene(s) selected from the group consisting of: CD45, CD2, CD5, CD7, CD4, and CD8.

In another embodiment the invention relates to a method as described above wherein the immune effector cell of (c) is a cell which expresses one or more gene(s) selected from the group consisting of: CD3, CD45, CD2, CD5, CD7, CD4, and CD8.

In another embodiment the invention relates to a method as described above wherein the immune effector cell of (c) is a cell which expresses CD3 (e.g. CD3epsilon/CD3e).

In another embodiment the invention relates to a method as described above wherein the immune effector cell of (c) is a cell which expresses one or more gene(s) selected from the list consisting of: CD105 and CD73 at a lower level compared to the expression of the same gene(s) in a vertebrate somatic cell of (a).

In another embodiment the invention relates to a method as described above wherein the immune effector cell of (c) is a cell which does not express one or more gene(s) selected from the list consisting of: CD105 and CD73.

In another embodiment the invention relates to a method as described above wherein the immune effector cell of (c) is a cell which has a round, lymphocyte morphology.

In another embodiment the invention relates to a method as described above wherein inducing expression of at least three transcription factors comprises: providing nucleic acid(s) comprising three nucleotide sequences, each nucleotide sequence encoding a transcription factor selected from said at least three transcription factors, each nucleotide sequence being operatively linked to a promoter sequence capable of directing expression of said transcription factor; and introducing said nucleic acid into said cell.

In another embodiment the invention relates to a method as described above wherein introducing said nucleic acid into said cell comprises electroporation of said cell.

In another embodiment the invention relates to a cell obtained by the method as described above.

In another embodiment the invention relates to an isolated nucleic acid comprising nucleotide sequences encoding Tbet, Ets1 and Tcf7, each nucleotide sequence being operatively linked to a promoter sequence capable of directing expression of said nucleotide sequence encoding Tbet, Ets1 and Tcf7.

In another embodiment the invention relates to an isolated nucleic acid as described above, wherein each said nucleotide sequence encoding Tbet, Ets1 and Tcf7 is operatively linked to a respective promoter sequence.

In another embodiment the invention relates to an isolated nucleic acid as described above, wherein said nucleotide sequences encoding Tbet, Ets1 and Tcf7 are all operatively linked to a single promoter sequence.

Suitably said nucleotide sequences are arranged in the order 5′-promoter sequence-nucleotide sequence encoding Tbet-nucleotide sequence encoding Ets1-nucleotide sequence encoding Tcf7-3′.

In another embodiment the invention relates to an isolated ribonucleic acid (RNA) comprising ribonucleotide sequences encoding Tbet, Ets1 and Tcf7 wherein said ribonucleotide sequences are arranged in the order 5′-ribonucleotide sequence encoding Tbet-ribonucleotide sequence encoding Ets1-ribonucleotide sequence encoding Tcf7-3′.

In another embodiment the invention relates to use of an isolated nucleic acid as described above for inducing transdifferentiation of a vertebrate somatic cell to an immune effector cell.

In another embodiment the invention relates to a pharmaceutical composition comprising a cell as described above or an isolated nucleic acid as described above.

In another embodiment the invention relates to a method of treating a subject comprising administering to said subject a cell as described above or an isolated nucleic acid as described above, or a pharmaceutical composition as described above.

Suitably the transcription factor comprises, or is, a ubiquitous transcription factor.

Suitably the transcription factor comprises, or is, a chromatin structure modifier transcription factor.

In one aspect, the invention relates to a method of treating a subject comprising administering to said subject a cell as described above or a pharmaceutical composition as described above.

Suitably treatment is treatment of cancer.

Suitably treatment is treatment of a tumour.

Suitably the method as described above comprises inducing expression of the transcription factor Tbet.

### DETAILED DESCRIPTION OF THE INVENTION

The following terms are used as in the art (e.g. Eguizabal et al. 2013 Semin Reprod Med Volume 31, pages 82 to 91):
Dedifferentiation refers to a process where differentiated cells are converted into progenitor cells.
Transdifferentiation refers to the conversion of cells from one differentiated type into another differentiated type.
Reprogramming refers to the induction of somatic cells to become pluripotent cells such as pluripotent stem cells (iPSC).

‘CD’ means ‘cluster of differentiation’ and is a surface marker that identifies a particular differentiation lineage.

The inventors describe an approach whereby somatic cells which are capable of in vitro expansion are directly transdifferentiated into immune effector cells, for example effector T-cells, optionally effector CAR T-cells.

The selection scheme was specifically designed by the inventors as a result of significant intellectual effort, and is evidence of the inventiveness (non-obviousness) of the invention. In this regard we refer to FIG. 4A/4B/4C which show the Bcl11b reporter-based screening platform; and in particular FIG. 4A which shows the Bcl11b-mCherry MSCs and MEFs as screening platform. The selection strategy was further developed when examining the pool of 62 transcription factors which was large and unwieldy and the inventors further designed strategies to select and/or optimise the pools of transcription factors analysed in order to design pools which achieved the desired novel effect and also to eliminate unnecessary factors to simplify and streamline the invention. A range of strategies were designed to address this further problem including subtractive, pooling and direct selection approaches. In addition, it should be noted that the transcription factors discussed herein each have very diverse tissue-restricted expression patterns and this is further evidence of the cryptic and challenging nature of the invention.

In practice, transdifferentiation may have sub-steps such as dedifferentiation and/or cell division before differentiation into the final state. In this case, the process is still correctly referred to as transdifferentiation—this refers to the switching to another differentiated cell type—which is a focus of the invention.

In one aspect the invention relates to transdifferentiation of expandable somatic cells to immune effector cells. Suitably the final cell or destination cell is an immune effector cell.

In one aspect the invention relates to transdifferentiation of expandable somatic cells to engineered immune effector cells. Suitably the final cell or destination cell is an engineered immune effector cell.

In one aspect the invention relates to transdifferentiation of expandable somatic cells to cytolytic immune cells. Suitably the final cell or destination cell is a cytolytic immune cell.

Suitably immune effector cells may be T cells. Suitably immune effector cells may be engineered to express a CAR or transgenic TCR.

Such engineering may be carried out at any appropriate stage e.g. carried out on the somatic cell (starting cell or source cell) or carried out on the final cell or destination cell, for example the immune effector cell created by the method.

‘Effector cells’ (effector T-cells) refers to the superset of all the various T cell types that actively respond to a stimulus, such as co-stimulation. Thus ‘effector cells’ may include helper, killer, and regulatory T cell types.

T helper cells (T_{H} cells) are involved in (among other things) activation of cytotoxic T cells and macrophages. T helper cells are also known as CD4+ T cells because they express the CD4 glycoprotein on their surfaces. Cytotoxic T cells (“Killer T cells” or “CTLs”) destroy target cells such as virus-infected cells or tumour cells. Cytotoxic T cells are also known as CD8+ T cells because they express the CD8 glycoprotein on their surfaces.

Suitably the T-cell may be an alpha-beta T cell or a gamma-delta T cell. As is well known in the art, these designations refer to the composition of the TCR expressed by the cell. Suitably the TCR may be αβ or γδ.

The final cell or destination cell may comprise a cytolytic immune cell such as a T cell.

In more detail, T cells or T lymphocytes are a type of lymphocyte that play a central role in cell-mediated immunity. They can be distinguished from other lymphocytes, such as B cells and natural killer cells (NK cells), by the presence of a T-cell receptor (TCR) on the cell surface. There are various types of T cell, as summarised below.

Helper T helper cells (TH cells) assist other white blood cells in immunologic processes, including maturation of B cells into plasma cells and memory B cells, and activation of cytotoxic T cells and macrophages. TH cells express CD4 on their surface. TH cells become activated when they are presented with peptide antigens by MHC class II molecules on the surface of antigen presenting cells (APCs). These cells can differentiate into one of several subtypes, including TH1, TH2, TH3, TH17, Th9, or TFH, which secrete different cytokines to facilitate different types of immune responses.

Cytolytic T cells (TC cells, or CTLs) destroy virally infected cells and/or tumour cells, and are also implicated in transplant rejection. CTLs express CD8 at their surface. These cells recognise their targets by binding to antigen associated with MHC class I, which is present on the surface of all nucleated cells. Through IL-10, adenosine and other molecules secreted by regulatory T cells, the CD8+ cells can be inactivated to an anergic state, which prevents autoimmune diseases such as experimental autoimmune encephalomyelitis.

Memory T cells are a subset of antigen-specific T cells that persist long-term after an infection has resolved. They quickly expand to large numbers of effector T cells upon re-exposure to their cognate antigen, thus providing the immune system with “memory” against past infections. Memory T cells comprise three subtypes: central memory T cells (TCM cells) and two types of effector memory T cells (TEM cells and TEMRA cells). Memory cells may be either CD4+ or CD8+. Memory T cells typically express the cell surface protein CD45RO.

Regulatory T cells (Treg cells), formerly known as suppressor T cells, are crucial for the maintenance of immunological tolerance. Their major role is to shut down T cell-mediated immunity toward the end of an immune reaction and to suppress auto-reactive T cells that escaped the process of negative selection in the thymus.

Two major classes of CD4+ Treg cells have been described—naturally occurring Treg cells and adaptive Treg cells.

Naturally occurring Treg cells (also known as CD4+CD25+FoxP3+Treg cells) arise in the thymus and have been linked to interactions between developing T cells with both myeloid (CD11c+) and plasmacytoid (CD123+) dendritic cells that have been activated with TSLP. Naturally occurring Treg cells can be distinguished from other T cells by the presence of an intracellular molecule called FoxP3. Mutations of the FOXP3 gene can prevent regulatory T cell development, causing the fatal autoimmune disease IPEX. Adaptive Treg cells (also known as Tr1 cells or Th3 cells) may originate during a normal immune response.

Suitably the processes/methods invention deliver transdifferentiation of the starting cells to immune effector cells. More suitably the processes/methods invention deliver transdifferentiation of the starting cells to effector T cells. More suitably the processes/methods invention deliver transdifferentiation of the starting cells to CD4+ T cells. More suitably the processes/methods invention deliver transdifferentiation of the starting cells to CD8+ T cells.

In a broad aspect the invention relates to a method of generating a CAR or TCR immune effector cell by means of trans-differentiation from another cell type.

Suitably the starting cell or source cell is a vertebrate somatic cell.

Suitably the starting cell or source cell is an expandable vertebrate somatic cell.

Suitably the starting cell or source cell is a mesenchymal stem cell.

Suitably the starting cell or source cell is, or is derived from, a skin fibroblast.

Suitably the starting cell or source cell is, or is derived from, a keratinocyte.

Suitably the starting cell or source cell is, or is derived from, a hepatocyte.

Suitably the starting cell or source cell is, or is derived from, an endothelial cell, such as a vascular endothelial cell. Suitably said endothelial cell is harvested e.g. from a cord.

The term “expandable” as in “expandable vertebrate somatic cell” has its normal meaning in the art i.e. that the cell can be maintained and/or induced to proliferate (i.e. expand in number).

Suitably the final cell or destination cell is an alpha/beta T-cell.

Suitably the final cell or destination cell is a gamma/delta T-cell.

Suitably the cell is genetically modified or engineered before being transdifferentiated.

In one embodiment suitably the genetic modification or engineering comprises transduction with a retroviral or lentiviral vector to introduce a transgene.

In one embodiment suitably the genetic modification or engineering comprises transposition to introduce a transgene.

In one embodiment suitably the genetic modification or engineering comprises homologous recombination to introduce a transgene

In one embodiment suitably the genetic modification or engineering comprises genomic editing by means of zinc finger nucleases, TALENs, megaTALENS and/or CrispR/CAS9.

In one embodiment suitably the transgene(s) comprise coding sequence for one or more transcription factors as described herein, together with nucleotide sequence(s) capable of directing their expression in the host cell.

In one embodiment suitably the transgene(s) comprise coding sequence for one or more of CAR, TCR, TCR kdel, Macho CAR, or dTBRII as described herein, together with nucleotide sequence(s) capable of directing their expression in the host cell.

Transdifferentiation

Transdifferentiation typically involves expression of transcription factors which allow transformation of a cell from one type to another. The transcription factors are typically expressed transiently. This transcription factor expression is most conveniently achieved by electroporation with mRNA encoding the transcription factor(s). The transcription factor(s) may be expressed in combination. The transcription factor(s) may be expressed at different times.

The transdifferentiation may be supported by one or more small molecule(s) and/or cytokine(s) and/or ligand(s). These may be supplied in the medium in which the cells are incubated, and/or may be supplied by directing their expression in said cells.

Genetic Modification

Optionally the cells may be genetically modified so that the final transdifferentiated cell (sometimes referred to as the ‘final’ cell or the ‘destination’ cell), for example the immune effector cell, comprises certain useful genetic elements. This genetic modification may be referred to as ‘engineering’.

For example a small stock of vertebrate somatic cells such as MSCs may be engineered on a small scale as desired. Such engineering might include introduction of CAR gene(s), introduction of suicide gene(s), introduction of enhancement factor(s), introduction of factor(s) which prevent rejection of engineered cells.

Engineering may include genome editing.

Examples of engineering are shown below.

| Example of Genetic | |
|---|---|
| Modification | Description/Utility |
| Introduction of CAR | To re-direct T-cells to a surface antigen target |
| Introduction of TCR | To re-direct T-cells to a peptide presented |
| | on MHC |
| TCR knock out | To prevent possibility of GvHD |
| Introduction of TCR kdel | To prevent possibility of GvHD |
| B2M ko | To prevent allo-rejection by recipient |
| Macho CAR approaches | To prevent allo-rejection by recipient |
| dSHP2 | To prevent inhibition by check-point |
| | inhibitory signals |
| Introduction of dTBRII | To prevent inhibition by TGFbeta |
| Knockout of PD1 | To prevent inhibition by PDL1 |
| Knockout of TGFBeta | To prevent inhibition by TGFbeta |
| receptor | |
| Other desired | |
| modification(s) as chosen | |
| by operator | |

Suitably a genetic modification is introduction of a CAR.

Suitably the method comprises a step of:
introducing to said cell a nucleic acid comprising nucleotide sequence encoding one or more elements of a chimeric antigen receptor (CAR).

Suitably the method comprises a step of:
introducing to said cell a nucleic acid comprising nucleotide sequence encoding a chimeric antigen receptor (CAR).

CAR T-Cells

AT-cell is a ‘CAR T-cell’ if it comprises a chimeric antigen receptor (CAR). The invention principally describes transdifferentiation of somatic cells such as expandable somatic cells into immune effector cells, for example T-cells such as effector T-cells. Also described is optional genetic modification to introduce one or more CAR(s) into the cells. This may be done either before, during or after the steps necessary for transdifferentiation. Thus, the main product of the method of the invention is an immune effector cell. When optional genetic modification step(s) are also carried out at appropriate point(s) in the transdifferentiation method, the product of the method of the invention will be an effector CAR T-cell. Thus the invention relates to transdifferentiation into an immune effector cell, for example a T-cell such as an effector T-cell, and does not necessarily require genetic modification to introduce one or more CARs—this is an optional step/steps in a preferred embodiment of the invention as discussed below.

It can be advantageous to introduce a chimeric antigen receptor (CAR) into the cells as part of the manipulation/method of the invention. Thus in one embodiment the invention relates to transdifferentiation and genetic modification of expandable somatic cells to immune effector CAR T-cells. The genetic modification may be optionally carried out at any suitable point in the transdifferentiation method. The genetic modification to add a CAR is not an essential step in the transdifferentiation method. Thus the method of the invention does not necessarily require a genetic modification to introduce a CAR. However, T-cells bearing a CAR have additional utility/industrial applications as treatment(s) in some haematological malignancies and may have wide applications as a cancer treatment. Therefore in some embodiments the invention involves optional genetic modification to introduce one or more CAR's into the cell of interest.

### Cells

Unless otherwise apparent from the context, references to ‘cell’ or ‘cells’ herein apply to the starting cell(s) upon which the methods of the invention are carried out (sometimes referred to as ‘source’ cell(s)). In other words, these cell(s) are suitably the cell(s) which are transdifferentiated into immune effector cells, for example effector T-cell(s), such as effector CAR T-cells.

For the avoidance of doubt, the transdifferentiated cell which results from the method of the invention may sometimes be referred to as the ‘destination’ cell or ‘final’ cell.

Thus in one embodiment the invention may be expressed as a method comprising
(a) providing a starting cell which is a vertebrate somatic cell
(b) inducing expression in said cell of at least three transcription factors, wherein said transcription factors are:
   i. Tbet (Tbx21),
   ii. Tcf7, and
   iii. Ets1; and
(c) incubating said cell to allow transdifferentiation;
wherein said cell is transdifferentiated to a destination cell or final cell, which is an immune effector cell.

Suitably the cell is a cell from a eukaryotic organism having an adaptive immune system. Suitably the cell is a vertebrate cell. Suitably the cell is a mammalian cell. Suitably the cell is a human cell.

In one embodiment suitably the invention does not relate to a method of medical treatment. In this embodiment suitably the cell is an in vitro cell.

Suitably the cell is not from a human embryo. Suitably the cell is not a germ line cell. Suitably the cell is a somatic cell.

Suitably the cell is an in vitro cell or an ex-vivo cell. More suitably the cell is an in vitro cell. The in vitro cell may find industrial application in treatment of disease. In one embodiment the invention relates to the use of a cell as described above in medicine. In one embodiment the invention relates to the use of a cell as described above in the preparation of a medicament for treatment of cancer. In one embodiment the invention relates to the use of a cell as described above in the treatment of cancer. In one embodiment the invention relates to a pharmaceutical composition comprising a cell as described above.

In one embodiment suitably the method does not involve direct medical intervention on the human or animal body; suitably the method is non-invasive; suitably the method is an in vitro method. In this regard, suitably the cell in an in vitro cell. Suitably the step of providing the cell comprises providing an in vitro cell. Suitably the cell is not derived directly from a subject such as a human subject, but suitably is incubated or cultured in vitro such that provision of the cell is provision of an in vitro cell and does not require presence of the human or animal body.

In one embodiment, the invention may be practised on the human or animal body; in this embodiment the cell may be provided by directly obtaining said cell from the human or animal body and practising the method of the invention using that cell as the starting cell.

Suitably the somatic cell is of a somatic cell type which is susceptible to expansion in vitro. Suitably the somatic cell is of a somatic cell type which is easy to expand, such as easy to expand in profusion. Somatic cell types which can be easily expanded include mesenchymal stem cells and/or skin fibroblasts. Thus suitably the somatic cell is selected from the group consisting of: a mesenchymal stem cell and a skin fibroblast. Suitably the somatic cell is a mesenchymal stem cell. Suitably the somatic cell is a skin fibroblast.

An advantage of this approach is that large numbers of immune effector cells can be generated using the simple process/method of the invention. Another advantage is that this allows production and/or expansion of immune effector cells, for example T-cells, such as CAR T-cells, from one donor.

The starting cells are suitably easily expanded from a normal donor.

Suitably exemplary starting cells may be:
Mesenchymal stem cells;
Skin fibroblasts;
Vascular endothelial cells; or
Keratinocytes.

More suitably exemplary starting cells may be:
Bone marrow derived mesenchymal stem cells;
Skin fibroblasts derived from skin biopsy or foreskin;
Vascular endothelial cells derived from cord; or
Keratinocytes derived from hair follicles.

Thus in one embodiment the invention provides a method as described above wherein the vertebrate somatic cell is selected from the group consisting of: a mesenchymal stem cell (MSC, preferably a human mesenchymal stem cell (hMSC)), a skin fibroblast, a vascular endothelial cell, and a keratinocyte.

More suitably the invention provides a method as described above wherein the vertebrate somatic cell is selected from the group consisting of: Bone marrow derived mesenchymal stem cells; Skin fibroblasts derived from skin biopsy or foreskin; Vascular endothelial cells derived from cord; and Keratinocytes derived from hair follicles.

Most suitably the vertebrate somatic cell is a human mesenchymal stem cell (hMSC), wherein said hMSC is derived from bone marrow.

Isolation of MSCs is routine. This technique is well within the ambit of the skilled worker. For example we refer to by Bieback et al 2008 Transfus. Med. Hemother. volume 35 pages 286-294 which describes clinical protocols for the isolation and expansion of Mesenchymal Stromal Cells; this document is incorporated herein by reference specifically for the teaching of the isolation and harvest of MSCs, including their expansion as necessary.

Suitably bone marrow derived mesenchymal stem cells are generated using standard process from a bone-marrow aspirate. This may be as described in Gnecchi and Melo 2009 (Methods Mol Biol. 2009; volume 482, pages 281-94) which describes in detail bone marrow-derived mesenchymal stem cells, their isolation, expansion, characterisation, viral transduction, and production of conditioned medium and is incorporated herein by reference. This document uses mouse as an example but the person skilled in the art can make any adaptations needed for application to human cells.

Alternative starting cells may include a semi-immortalized cell line derived from an embryo.

Alternative starting cells may include an immortalized cell line.

Human Mesenchymal Stem Cells (hMSCs)

Suitably the vertebrate somatic cell (or starting cell or source cell) is a mesenchymal stem cell (MSC), more suitably a human mesenchymal stem cell (hMSC).

Production of hMSCs, such as their isolation from different tissues, is well known in the art. hMSCs may be isolated from a range of tissues including adipose tissue, amniotic fluid, endometrium, dental tissues, umbilical cord and/or Wharton's jelly.

The International Society for Cellular Therapy has proposed criteria to define MSCs.

In particular, the Mesenchymal and Tissue Stem Cell Committee of the International Society for Cellular Therapy proposes minimal criteria to define human MSCs:
1) MSC must be plastic-adherent when maintained in standard culture conditions;
2a) MSC must express CD105, CD73 and CD90;
2b) MSC must lack expression of CD45, CD34, CD14 or CD11b, CD79alpha or CD19 and HLA-DR surface molecules; and
3) MSC must differentiate to osteoblasts, adipocytes and chondroblasts in vitro.

When the starting cell is a MSC, optionally a stock of MSCs may be generated.

Once a MSC stock is generated, optionally the MSCs may be expanded to large numbers for example in a biobank.

In order to provide a vertebrate somatic cell, suitably MSCs are expanded until sufficient in number. The operator will choose the sufficient number depending on how many cells they want to end up with.

To induce expression of at least one transcription factor, MSCs are electroporated (e.g. using a large scale electroporator) with synthetic mRNA which encodes and is capable of expressing the required transcription factor or combination of transcription factors.

Electroporated MSCs are incubated (e.g. cultivated) in T-cell supportive medium.

Suitably their phenotype is checked using flow-cytometry.

Optionally cell sorting either by magnetic beads, or by clinical grade flow sorting, may be used to isolate cells of the desired phenotype.

Optionally cells are aliquoted in appropriate cell numbers (e.g. doses for administration).

Optionally cells are cryopreserved.

Cell Media

The skilled worker can select appropriate media for the cells being used in the invention. For example, MSCs may be cultured in Dulbecco's Modified Eagle Medium (DMEM) or a suitable variant thereof. Supplementation with foetal bovine serum/foetal calf serum (FBS/FCS) is well known in the art. Various minimal media such as Minimal Essential Medium Eagle (MEM) or other variants such as High Glucose DMEM (HGDMEM), low glucose DMEM (DMEMLG) or alpha-DMEM may be used as appropriate.

Suitably cells in which transcription factor(s) have been induced are incubated in T-cell supportive medium. The composition(s) of T-cell supportive medium is well known in the art, (for example as described by Cell Culture Dish, Inc. of 1112 Oakridge Drive Ste 104 PMB 259 Fort Collins, CO 80525, USA, from which the following guidance is adapted:

At a minimum, T cell medium includes a buffer system, protein, trace elements, vitamins, inorganic salts, and energy sources. Many formulations contain or require addition of IL-2, a cytokine important for T cell expansion. Perhaps the most commonly used media for T cell expansion is RPMI 1640 basal medium supplemented with 10% fetal bovine serum (FBS). While FBS is a robust additive supporting the culture of many different cell types, its inclusion in culture media may be undesirable for certain applications. For T cell culture, there has been a shift in the field towards animal-free, more defined media formulations with better consistency, traceability and regulatory compliance as researchers move forward with an eye towards cell therapy. In the world of cell culture media, there are standard terms used to define the level of purity of the ingredients, though often these terms are misunderstood or misused. For instance, xeno-free media is gaining ground in the T cell field. Xeno-free indicates that the product does not contain non-human components. Unfortunately, FBS, the gold standard media supplement, is non-human, and several xeno-free serum replacements are available on the market now. Chemically-defined media provides some advantages, namely that the composition and concentration of all the components are known. However, the tradeoff is that cells often do not grow as well in these more defined, less robust conditions. For adoptive T cell therapy, patient-derived T cells failed to grow optimally in serum-free media and exhibited reduced efficacies of gene transfer resulting from suboptimal T cell activation, which is likely not an acceptable practice. Therefore, the skilled worker will evaluate their needs prior to selecting a media type when working the invention.

RPMI medium is a standard cell culture medium. RPMI is an abbreviation of “RPMI 1640” (named after the Roswell Park Memorial Institute where it was formulated). RPMI is widely available from numerous suppliers worldwide and contains (per litre): Glucose (2 g), pH indicator (phenol red, 5 mg), Salts (6 g sodium chloride, 2 g sodium bicarbonate, 1.512 g disodium phosphate, 400 mg potassium chloride, 100 mg magnesium sulfate, and 100 mg calcium nitrate), Amino acids (300 mg glutamine; 200 mg arginine; 50 mg each asparagine, cystine, leucine, and isoleucine; 40 mg lysine hydrochloride; 30 mg serine; 20 mg each aspartic acid, glutamic acid, hydroxyproline, proline, threonine, tyrosine, and valine; 15 mg each histidine, methionine, and phenylalanine; 10 mg glycine; 5 mg tryptophan; and 1 mg reduced glutathione), plus vitamins (35 mg i-inositol; 3 mg choline chloride; 1 mg each para-aminobenzoic acid, folic acid, nicotinamide, pyridoxine hydrochloride, and thiamine hydrochloride; 0.25 mg calcium pantothenate; 0.2 mg each biotin and riboflavin; and 0.005 mg cyanocobalamin).

Any supplementation will be at the choice of the skilled worker as required.

Suitably the T-cell supportive medium may be obtained from Nucleus Biologics, 10929 Technology Place, San Diego, CA 92127, USA.

Most suitably the T-cell supportive medium is RPMI supplemented with L-glutamine, human protein lysate and IL7/IL15.

Transcription Factors

Suitably the methods of the invention involve induction of, or expression of, certain transcription factor(s) in the cells of interest.

Relevant, or additional, transcription factors may be identified by in silico techniques. In one embodiment the method requires inducing expression of at least three transcription factors, wherein said transcription factors are:
i. Tbet (Tbx21),
ii. Tcf7, and
iii. Ets1.

As shown in FIG. 2, some transcription factors are already highly expressed in MSCs. Suitably the transcription factors comprise, or are, transcription factor(s) shown in FIG. 2.

NFATc Family

NFATc family transcription factors represent a group. The NFATc family has 4-5 proteins that all play some roles in T cell development.

In more detail, the NFAT family consists of five members: NFAT1 (also known as NFATp or NFATc2), NFAT2 (also known as NFATc or NFATc1), NFAT3 (also known as NFATc4), NFAT4 (also known as NFATx or NFATc3) and NFAT5. This family and the functions and mechanisms of action of the individual proteins, including the roles of NFAT family in T cell development, is reviewed in Macian 2005 (Nature Reviews Immunology, vol. 5, pages 472-484).

Among them NFATc3 (NFAT4) plays the most important role. NFAT4 KO mice will “have Impaired development of CD4 and CD8 single-positive cells, with increased apoptosis of double-positive thymocytes. Also mild hyperactivation of peripheral T cells”. NFAC1 (NFAT2) and NFAC2(NFAT2) also play some moderate roles in T cell development.

Suitably the transcription factors comprise, or are, NFAT1, NFAT2, NFAT3, NFAT4, or NFAT5.

More suitably the transcription factors comprise, or are, NFATc3 (NFAT4).

TCF1/7

TCF7 is occasionally referred to by its alias TCF1 (TCF1 is also alias for some different genes in other fields of research). For the avoidance of doubt, mention of TCF7 or TCF1 herein refers to transcription factor TCF7 which is the official and unique gene symbol now. The accession number (reference sequence) for TCF7 is provided in the table herein.

Induction

Suitably induction of expression of transcription factor(s) may be by manipulation of the cell so that it expresses the one or more transcription factor(s) from its existing repertoire of genes.

Alternatively induction of expression may be by introduction of a nucleic acid into the cell which directs expression of said one or more transcription factor(s) from said nucleic acid.

In one embodiment suitably the nucleic acid which directs expression of said one or more transcription factor(s) comprises nucleotide sequence encoding said transcription factor(s) under the control of a promoter capable of directing expression in the vertebrate somatic cell.

Suitably the promoter is operatively linked to the coding sequence. Suitably the promoter is on the same nucleic acid as the coding sequence.

Examples of promoters suitable for use in directing expression of transcription factors as taught herein include:

| | Exemplary Sequence |
|---|---|
| Promoter | (Reference Sequence) |
| SEQ ID NO: 1 | CGTTACATAACTTACGGTAAATGGCCCGCC |
| human | TGGCTGACCGCCCAACGACCCCCGCCCATT |
| cytomegalovirus | GACGTCAATAATGACGTATGTTCCCATAGT |
| promoter | AACGCCAATAGGGACTTTCCATTGACGTCA |
| such as the | ATGGGTGGAGTATTTACGGTAAACTGCCCA |
| long CMV | CTTGGCAGTACATCAAGTGTATCATATGCC |
| promoter, or | AAGTACGCCCCCTATTGACGTCAATGACGG |
| cytomegalovirus | TAAATGGCCCGCCTGGCATTATGCCCAGTA |
| (CMV) immediate- | CATGACCTTATGGGACTTTCCTACTTGGCA |
| early enhancer- | GTACATCTACGTATTAGTCATCGCTATTAC |
| promoter | CATGGTGATGCGGTTTTGGCAGTACATCAA |
| | TGGGCGTGGATAGCGGTTTGACTCACGGGG |
| | ATTTCCAAGTCTCCACCCCATTGACGTCAA |
| | TGGGAGTTTGTTTTGGCACCAAAATCAACG |
| | GGACTTTCCAAAATGTCGTAACAACTCCGC |
| | CCCATTGACGCAAATGGGCGGTAGGCGTGT |
| | ACGGTGGGAGGTCTATATAAGCAGAGCT |
| SEQ ID NO: 2 | CGTGAGGCTCCGGTGCCCGTCAGTGGGCAG |
| human EF1alpha | AGCGCACATCGCCCACAGTCCCCGAGAAGT |
| promoter | TGGGGGGAGGGGTCGGCAATTGAACCGGTG |
| | CCTAGAGAAGGTGGCGCGGGGTAAACTGGG |
| | AAAGTGATGTCGTGTACTGGCTCCGCCTTT |
| | TTCCCGAGGGTGGGGGAGAACCGTATATAA |
| | GTGCAGTAGTCGCCGTGAACGTTCTTTTTC |
| | GCAACGGGTTTGCCGCCAGAACACAGGTAA |
| | GTGCCGTGTGTGGTTCCCGCGGGCCTGGCC |
| | TCTTTACGGGTTATGGCCCTTGCGTGCCTT |
| | GAATTACTTCCACGCCCCTGGCTGCAGTAC |
| | GTGATTCTTGATCCCGAGCTTCGGGTTGGA |
| | AGTGGGTGGGAGAGTTCGAGGCCTTGCGCT |
| | TAAGGAGCCCCTTCGCCTCGTGCTTGAGTT |
| | GAGGCCTGGCCTGGGCGCTGGGGCCGCCGC |
| | GTGCGAATCTGGTGGCACCTTCGCGCCTGT |
| | CTCGCTGCTTTCGATAAGTCTCTAGCCATT |
| | TAAAATTTTTGATGACCTGCTGCGACGCTT |
| | TTTTTCTGGCAAGATAGTCTTGTAAATGCG |
| | GGCCAAGATCTGCACACTGGTATTTCGGTT |
| | TTTGGGGCCGCGGGCGGCGACGGGGCCCGT |
| | GCGTCCCAGCGCACATGTTCGGCGAGGCGG |
| | GGCCTGCGAGCGCGGCCACCGAGAATCGGA |
| | CGGGGGTAGTCTCAAGCTGGCCGGCCTGCT |
| | CTGGTGCCTGGCCTCGCGCCGCCGTGTATC |
| | GCCCCGCCCTGGGCGGCAAGGCTGGCCCGG |
| | TCGGCACCAGTTGCGTGAGCGGAAAGATGG |
| | CCGCTTCCCGGCCCTGCTGCAGGGAGCTCA |
| | AAATGGAGGACGCGGCGCTCGGGAGAGCGG |
| | GCGGGTGAGTCACCCACACAAAGGAAAAGG |
| | GCCTTTCCGTCCTCAGCCGTCGCTTCATGT |
| | GACTCCACGGAGTACCGGGCGCCGTCCAGG |
| | CACCTCGATTAGTTCTCGAGCTTTTGGAGT |
| | ACGTCGTCTTTAGGTTGGGGGGAGGGGTTT |
| | TATGCGATGGAGTTTCCCCACACTGAGTGG |
| | GTGGAGACTGAAGTTAGGCCAGCTTGGCAC |
| | TTGATGTAATTCTCCTTGGAATTTGCCCTT |
| | TTTGAGTTTGGATCTTGGTTCATTCTCAAG |
| | CCTCAGACAGTGGTTCAAAGTTTTTTTCTT |
| | CCATTTCAGGTGTCGTGAG |
| SEQ ID NO: 3 | CCACGGGGTTGGGGTTGCGCCTTTTCCAAG |
| Human PGK | GCAGCCCTGGGTTTGCGCAGGGACGCGGCT |
| promoter | GCTCTGGGCGTGGTTCCGGGAAACGCAGCG |
| | GCGCCGACCCTGGGTCTCGCACATTCTTCA |
| | CGTCCGTTCGCAGCGTCACCCGGATCTTCG |
| | CCGCTACCCTTGTGGGCCCCCCGGCGACGC |
| | TTCCTGCTCCGCCCCTAAGTCGGGAAGGTT |
| | CCTTGCGGTTCGCGGCGTGCCGGACGTGAC |
| | AAACGGAAGCCGCACGTCTCACTAGTACCC |
| | TCGCAGACGGACAGCGCCAGGGAGCAATGG |
| | CAGCGCGCCGACCGCGATGGGCTGTGGCCA |
| | ATAGCGGCTGCTCAGCAGGGCGCGCCGAGA |
| | GCAGCGGCCGGGAAGGGGCGGTGCGGGAGG |
| | CGGGGTGTGGGGCGGTAGTGTGGGCCCTGT |
| | TCCTGCCCGCGCGGTGTTCCGCATTCTGCA |
| | AGCCTCCGGAGCGCACGTCGGCAGTCGGCT |
| | CCCTCGTTGACCGAATCACCGACCTCTCTC |
| | CCCAGG |
| SEQ ID NO: 4 | GTAACGCCATTTTGCAAGGCATGGAAAAAT |
| Spleen focus | ACCAAACCAAGAATAGAGAAGTTCAGATCA |
| forming | AGGGCGGGTACATGAAAATAGCTAACGTTG |
| virus (SFFV) | GGCCAAACAGGATATCTGCGGTGAGCAGTT |
| promoter | TCGGCCCCGGCCCGGGGCCAAGAACAGATG |
| | GTCACCGCAGTTTCGGCCCCGGCCCGAGGC |
| | CAAGAACAGATGGTCCCCAGATATGGCCCA |
| | ACCCTCAGCAGTTTCTTAAGACCCATCAGA |
| | TGTTTCCAGGCTCCCCCAAGGACCTGAAAT |
| | GACCCTGCGCCTTATTTGAATTAACCAATC |
| | AGCCTGCTTCTCGCTTCTGTTCGCGCGCTT |
| | CTGCTTCCCGAGCTCTATAAAAGAGCTCAC |
| | AACCCCTCACTCGGCGCGCCAGTCCTCCGA |
| | CAGACTGAGTCGCCCGGGG |

A further example of a useful promoter is a CMV promoter as used in exemplary doxycycline-inducible lentiviral vector FUW-tetO-MCS (MCS=multiple cloning site i.e. empty vector). This vector—and therefore the promoter—is available from addgene (Addgene, LGC Standards, Teddington, UK) as deposit number/accession number 84008 (Plasmid #84008-RRID:Addgene_84008). In case any further guidance is needed, we refer to Panciera T, Azzolin L, Fujimura A, Di Biagio D, Frasson C, Bresolin S, Soligo S, Basso G, Bicciato S, Rosato A, Cordenonsi M, Piccolo S. Cell Stem Cell. 2016 Sep. 9. pii: S1934-5909(16)30256-9. In case any further information is required, we refer to SEQ ID NO: 8 which is the complete sequence of the vector. The exemplary promoter sequence may be easily taken from this complete sequence by the skilled reader.

A promoter is only needed for protocols where nucleic acid such as plasmid DNA is used to express the transcription factor. Suitably mRNA is used, which provides the advantage that there is no chance of stable integration.

Most suitably induction is carried out using synthetic mRNA which encodes the transcription factor(s). This mRNA is then introduced into the cell e.g. by electroporation. An advantage of this approach is that the mRNA only needs a cap structure, a kozak sequence and a polyA sequence together with the transcription factor coding sequence—it does not need a promoter. Further advantages of this mRNA approach include that the electroporation conditions can be milder for mRNA (compared with harsher conditions needed for good uptake of DNA). Further advantages of this mRNA approach include that there is no chance of stable insertion. SEQ ID NO: 7 provides exemplary RNA sequence encoding TBET-ETS1-TCF7. Addition of ancillary genetic elements such as those noted (e.g. polyA tail, cap, kozak sequence etc.) is within the ambit of the skilled reader.

It will be noted that some TFs are already expressed in MSCs. We refer to FIG. 2 which illustrates this. Thus, if the transcription factors whose expression is to be induced in the vertebrate somatic cell are already expressed at some level in said cell, then expression of said transcription factor(s) will be considered to have been induced if it is raised by at least 20%, more suitably at least 30%, more suitably at least 50%, more suitably at least 100% compared to the level of expression in the starting cell (source cell i.e. the vertebrate somatic cell before/without induction).

Suitably expression of said transcription factor(s) will be considered to have been induced if they are expressed many fold over physiological expression.

Suitably expression of said transcription factor(s) will be considered to have been induced if they are expressed 2 fold over physiological expression, more suitably 3 fold over physiological expression, more suitably 4 fold over physiological expression, more suitably 5 fold over physiological expression, more suitably 6 fold over physiological expression, more suitably 7 fold over physiological expression, more suitably 8 fold over physiological expression, more suitably 9 fold over physiological expression, more suitably 10 fold over physiological expression, or more. ‘Physiological expression’ may be regarded as the level of expression in the starting cell (source cell i.e. the vertebrate somatic cell before/without induction).

Timing

After performing the step(s) to induce expression of transcription factor(s), the cells are incubated to allow transdifferentiation to take place.

Suitably cells are incubated for 24-96 hours or more.

Suitably cells are incubated and split every two days until transdifferentiation takes place.

Suitably cells are provided with new/fresh medium at each split.

Suitably cells are provided with new/fresh medium every 48 hours.

Reference Sequences

Suitably all sequences herein are human unless otherwise apparent from the context.

Sequences deposited in databases can change over time. Suitably the current version of sequence database(s) are relied upon. Alternatively, the release in force at the date of filing is relied upon.

It may be helpful to refer to the GenBank sequence of the wild-type human gene(s)/promoter(s).

GenBank is a sequence database as described in Benson, D. et al, Nucleic Acids Res. 45(D1):D37-D42 (2017). In more detail, GenBank is as administered by the National Center for Biotechnology Information, National Library of Medicine, 38A, 8N805, 8600 Rockville Pike, Bethesda, MD 20894, USA. Suitably the current version of sequence database(s) are relied upon. Alternatively, the release in force at the date of filing is relied upon. For the avoidance of doubt, NCBI-GenBank Release 241 (15 Dec. 2020) is relied upon.

UniProt (Universal Protein Resource) is a comprehensive catalogue of information on proteins (‘UniProt: a hub for protein information’ Nucleic Acids Res. 43: D204-D212 (2015).). Suitably the current version of sequence database(s) are relied upon. Alternatively, the release in force at the date of filing is relied upon. For the avoidance of doubt, the UniProt consortium European Bioinformatics Institute (EBI), SIB Swiss Institute of Bioinformatics and Protein Information Resource (PIR)'s UniProt Knowledgebase (UniProtKB) Release 202101, (10 Feb. 2021) is relied upon.

When particular nucleotides/amino acid residues are referred to herein using numeric addresses, the numbering is taken with reference to the wild type sequence indicated by the accession numbers herein. This sequence is to be used as is well understood in the art to locate the feature/residue of interest. This is not always a strict counting exercise—attention must be paid to the context. For example, if the sequence of interest is of a slightly different length, then location of the correct nucleotide in that sequence may require the sequences to be aligned and the equivalent or corresponding nucleotide picked. This is well within the ambit of the skilled reader.

Mutating has it normal meaning in the art and may refer to the substitution or truncation or deletion or addition of one or more nucleotides, motifs or domains.

Suitably the gene designations of the transcription factors mentioned herein correspond to the reference sequences as given below.

| Transcription | Accession Number of Exemplary |
|---|---|
| Factor (Designation) | Sequence (Reference Sequence) |
| BCL11B | UniProt: Q9C0K0 |
| CBP | Q92793 |
| E2A | P15923 |
| E2F1 | Q01094 |
| E2F2 | Q14209 |
| EP300 | Q09472 |
| Ets1 | P14921 |
| GATA1 | P15976 |
| GATA3 | P23771 |
| HOXB5 | P09067 |
| Ikzf1 | Q13422 |
| Lef1 | Q9UJU2 |
| MYB | P10242 |
| NFATC3 | Q12968 |
| Nfil3 | Q16649 |
| Notch | P46531 |
| Nr4a3 | Q92570 |
| PU1 (SPI1) | P17947 |
| Rorc | P51449 |
| RUNX1 | Q01196 |
| RUNX3 | Q13761 |
| Satb1 | Q01826 |
| SPI1 | Q13761 |
| Stat4 | Q14765 |
| Tbet (Tbx21) | Q9UL17 |
| TCF7 | P36402 |
| Tcf12 (HEB) | Q99081 |
| Tox | O94900 |
| ZBTB7B (ThPOK) | O15156 |
| Zbtb16 | Q05516 |
| ZNF683 | Q8IZ20 |

In the event that any further guidance is required, most suitably the gene designations of the transcription factors mentioned herein correspond to the reference sequences as given below:

| Transcription | Accession Number of Exemplary |
|---|---|
| Factor (Designation) | Sequence (Reference Sequence) |
| BCL11B | UniProt: Q9C0K0 |
| CBP | Q92793 |
| E2A | P15923 |
| E2F1 | Q01094 |
| E2F2 | Q14209 |
| EP300 | Q09472 |
| GATA1 | P15976 |
| GATA3 | P23771 |
| HEB | Q99081 |
| HOXB5 | P09067 |
| MYB | P10242 |
| NFATC3 | Q12968 |
| Notch | P46531 |
| RUNX1 | Q01196 |
| RUNX3 | Q13761 |
| TCF7 (TCF1/TCF7) | P36402 |
| ZBTB7B (ThPOK) | O15156 |

In the event that the accession number/database entry discloses a sequence including more than the coding sequence, suitably only the coding sequence is referred to/relied upon.

As the skilled person knows, the accession numbers may be version/dated accession numbers. The citeable accession numbers for the current database entry are the same as above, but omitting the decimal point and any subsequent digits.

Optional Additional Method Step(s)

Cytokines/STATs/SMADs

One or more appropriate cytokines may be introduced to, or contacted with, the cell(s). Introduction of such cytokine(s) may be by induction of expression or by direct application.

Induction of expression may be by manipulation of the cell so that it expresses one or more cytokine(s) from its existing repertoire of genes. Alternatively induction of expression may be by introduction of a nucleic acid into the cell which directs expression of said one or more cytokine(s) from said nucleic acid.

Direct application may be by incorporation of said one or more cytokine(s) into the medium in which the cell(s) are incubated, or may be by addition of exogenous cytokine(s) to the medium at one or more specific step(s) and/or one or more specific timepoint(s), in the process of the invention.

One advantage of introducing to the cells, or contacting the cells with, one or more cytokine(s) as explained above is to activate STATs or SMADs.

Thus in one embodiment the method of the invention includes a step of activating one or more STAT(s) in said cell, and/or activating one or more SMAD(s) in said cell. Suitably a step of activating one or more STAT(s) in said cell, and/or a step of activating one or more SMAD(s) in said cell, comprises: introducing to the cells, or contacting the cells with, one or more cytokine(s).

Optionally appropriate cytokines may be added to the medium, or induced in the cells, (e.g. to activate STATs or SMADs), for example one or more of IL2, IL7, IL15, IL21.

Transcription Factor Repression

In some embodiments it may be desirable to repress B cell-specific and/or NK cell-specific TFs to promote transdifferentiation to T cells. These lineage-specific transcription factors usually cross-repress each other. Repressing or silencing B cell and/or NK cell TFs during reprogramming/transdifferentiation may advantageously enhance T cell production. Thus, in one embodiment optionally certain transcription factors may be repressed for commitment to T cell lineages, such as the transcription factors needed for B cell or NK cell lineages. In one embodiment suitably B cell TFs such as PAX5 (accession number Q02548) and EBF1 (accession number Q9UH73) are repressed. In one embodiment suitably NK cell TFs such as Nfil3 (accession number Q16649), ERG2 (accession number P11308) and PLZF (accession number Q05516) are repressed. In one embodiment both B cell TFs and NK cell TFs are repressed.

Suitably repression may be carried out before or after or at the same time as induction of transcription factor(s). Most suitably repression may be carried out at the same time as induction of transcription factors.

Transdifferentiation to an Immune Effector Cell

Suitably the methods of the invention result in the cell (i.e. the starting cell such as vertebrate somatic cell) being transdifferentiated to an immune effector cell.

Of course transdifferentiation can involve certain genes being switched off relative to the starting cell (such as vertebrate somatic cell) and certain genes being switched on relative to the starting cell (such as vertebrate somatic cell).

As is known in the art, for example as reviewed in Jopling et al. 2011, transdifferentiation can sometimes result in unnatural intermediate cells expressing genes characteristic of both the starting cell type and the finishing/destination cell type (i.e. the cell type which they are being transdifferentiated to). An example of this phenomenon in the prior art is the co-expression of B-cell specific genes (such as CD19) as well as macrophage specific genes (such as Maci) during transdifferentiation from B-cell to macrophage. Thus, the person skilled in the art will not necessarily expect all genes characteristic of the starting cell type to be fully switched off in order to acknowledge that transdifferentiation has taken place.

One way of checking whether the transdifferentiation (such as to an immune effector cell, for example an effector T cell) has been successful is to determine when morphological changes are evident that suggest T-cell trans-differentiation; examples of such morphological changes include round, lymphocyte morphology.

More suitably if a cell is CD2+CD5+CD7+ it is considered to be transdifferentiated to an effector immune cell.

More suitably if a cell is CD3+ it is considered to be transdifferentiated to an effector immune cell.

Effector immune cells such as T-cells may emerge from this process and may be a mixture of CD4+CD8−, CD4-CD8+ and CD4+CD8+. Thus most suitably if a cell is CD2+CD5+CD7+CD4+CD8−; or
CD2+CD5+CD7+CD4-CD8+; or
CD2+CD5+CD7+CD4+CD8+,
it is considered to be transdifferentiated to an effector immune cell.

Another way of checking whether the transdifferentiation (such as to an immune effector cell, for example an effector T-cell) has been successful is detection of expression of one or more of the following gene(s): CD45, CD2, CD5, CD7, CD4 and/or CD8.

Another way of checking whether the transdifferentiation (such as to an immune effector cell, for example an effector T-cell) has been successful is detection of expression of each of the genes in one of the groups set out below:
CD45, CD2, CD5, CD7, CD4 and CD8; or
CD45, CD2, CD5, CD7 and CD4; or
CD45, CD2, CD5, CD7 and CD8.

Optionally, and/or in addition to the positive expression of genes as explained above, downregulation, or absence of expression, of the following genes would also be indicative of successful transdifferentiation to an effector T-cell: classical markers of BM-derived MSCs—CD105 and CD73. Most suitably expression of CD105 and CD73 should be absent.

| Marker | Accession Number of Exemplary |
|---|---|
| Gene (Designation) | Sequence (Reference Sequence) |
| CD45 | P08575 |
| CD2 | P06729 |
| CD5 | P06127 |
| CD7 | P09564 |
| CD4 | P01730 |
| CD8 | P01732 (alpha chain) |
| | P10966 (beta chain) |
| CD105 | P17813 |
| CD73 | Q53Z63 |
| | Q6NZX3 |
| CD90 | P04216 |
| CD34 | P28906 |
| CD14 | P08571 |
| CD11b | P11215 |
| CD79alpha | P11912 |
| CD19 | P15391 |
| HLA-DR | P01903 (alpha chain) |
| CD3 | P07766 (CD3epsilon/CD3e) |

Obviously “absence” of expression has to be interpreted in a practical scientific manner. It is not the case that a single molecule of this gene would constitute “expression” of the gene—there are limits of detection with any analytical technique. Thus, suitably these genes are considered to be downregulated if they are expressed at a level lower than expression in the starting cell. Thus, a comparison may be made to the expression level in the starting cell and the expression level in the transdifferentiated cell, and if the expression level is lower in the transdifferentiated cell then the gene will be considered to be downregulated. If using the same detection method (including the same cut-offs/sensitivity as appropriate) expression of a gene is detected in the starting cell but is not detected in the transdifferentiated cell, then for all practical purposes this would be treated as an absence of expression in the differentiated cell.

Bcl11b Reporter Assay

The Bcl11b reporter assay is used herein to assess differentiation into the T-cell lineage.

The colony assays described are based on the number of Bcl11b-reporter positive colonies.

Bcl11b is a key driver of T-cell commitment and identity, being both expressed on T cell progenitors and effector cells and highly restricted to the T-cell lineage. We refer to FIGS. 4B and 4C which illustrate this. For an hematopoietic progenitor cell to differentiate into T-cell lineage it requires the irreversible activation of Bcl11b, blocking the differentiation into alternative cell types. In addition, the deletion of Bcl11b in T-cells leads to the loss of cell identity and their reprogramming to NK cells (Li et al. 2010 (‘Reprogramming of T cells to natural killer-like cells upon Bcl11b deletion.’ Science, 329, 85-9)). Bcl11b is critical to identify and maintain T-cell identity. In the direct reprogramming setting, the Bcl11b reporter system allows identification of cells that have committed to the T cell lineage.

Thus the significance of the Bcl11b positive colony assay is to show those cells which have committed to the T cell lineage.

In more detail, Bcl11b is specifically expressed on T cells and progenitors committed to the T cell lineage. Therefore, activation of Bcl11b reporter shows that the identified transcription factors (e.g. the ‘TET group’ described herein—i.e. TCF1, TBET and ETS1) are critical for T cell specification and to impose T cell program on fibroblasts.

### Further Embodiments

Embodiments of the invention are described by way of numbered paragraphs:

Paragraph 1. A method comprising
(a) providing a vertebrate somatic cell
(b) inducing expression in said cell of at least one transcription factor selected from the group consisting of:
   i. Tbx21(Tbet)
   ii. Tcf7
   iii. Ets1
   iv. BclIIb
   v. Gata3
   vi. Ikzf1
   vii. Satb1
   viii. RorC
   ix. Lef1; and
(c) incubating said cell to allow transdifferentiation; wherein said cell is transdifferentiated to an immune effector cell.

Paragraph 2. A method according to Paragraph 1 wherein
said at least one transcription factor is selected from the group consisting of:
   i. Tbx21 (Tbet)
   ii. Tcf7
   iii. Ets1
   iv. BclIIb
   v. Gata3
   vi. Ikzf1.

Paragraph 3. A method according to Paragraph 2 wherein
said at least one transcription factor is selected from the group consisting of:
   i. Tbx21 (Tbet)
   ii. Tcf7
   iii. Ets1
   iv. BclIIb.

Paragraph 4. A method according to Paragraph 1 or Paragraph 2 or Paragraph 3 wherein step (b) comprises
inducing expression in said cell of at least four transcription factors selected from said group.

Paragraph 5. A method according to Paragraph 4 comprising inducing expression of at least the four transcription factors in the group consisting of:
i. Tbx21 (Tbet)
ii. Tcf7
iii. Ets1
iv. BclIIb.

Paragraph 6. A method according to Paragraph 1 or Paragraph 2 wherein step (b) comprises
inducing expression in said cell of at least six transcription factors selected from said group.

Paragraph 7. A method according to Paragraph 6 comprising inducing expression of at least the six transcription factors in the group consisting of:
i. Tbx21 (Tbet)
ii. Tcf7
iii. Ets1
iv. BclIIb
v. Gata3
vi. Ikzf1.

Paragraph 8. A method according to any preceding numbered Paragraph comprising inducing expression of each of the transcription factors (i) to (ix).

In a broad embodiment the invention relates to a method comprising
(a) providing a vertebrate somatic cell
(b) inducing expression in said cell of at least three transcription factors selected from the group consisting of:
   i. Tbet (Tbx21)
   ii. Tcf7
   iii. Ets1
   iv. Bcl11b
   v. Satb1
   vi. Gata3
   vii. Lef1
   viii. Rorc
   ix. Ikzf1; and
(c) incubating said cell to allow transdifferentiation; wherein said cell is transdifferentiated to an immune effector cell.

### Advantages

It is an advantage of the invention that it may be applied to produce allogeneic T-cells and/or autologous T-cells depending on the starting material. Autologous T-cells are more desirable since they avoid issues of GvHD. Thus, suitably the starting cells are autologous with reference to the subject intended to receive the transdifferentiated T-cells.

In one embodiment the starting cells are allogeneic with reference to the intended or eventual recipient.

In one embodiment the starting cells are autologous with reference to the intended or eventual recipient.

Prior art approaches such as deriving iPSCs and then differentiating those iPSCs back to T-cells is a complex process. T-cell differentiation is a complex process involving step-wise progression through CD4/CD8 double-negative, double-positive and single-positive with TCR re-arrangement being an important part of the process. Typically, stromal cell lines which simulate the thymus are needed. The process takes many weeks. These are problems with this prior art approach. In contrast, the trans-differentiation approach of the invention is advantageous as it can be simpler and/or can allow the skilled worker to avoid or bypass these iPSC steps.

ESCs and iPSCs have nearly unlimited capacity to proliferate in culture. Using this known approach provides abundant starting material to produce desired cell types. However, a drawback is that the many cycles of proliferation required in the derivation of iPSCs and their subsequent differentiation may select for fast-growing, culture-adapted cells that harbour subtle genetic mutations. Such changes may result in unstable phenotypes, including cancer, when cells are transplanted in vivo, which is clearly a problem with known approaches using ESCs and/or iPSCs. In contrast, the trans-differentiation approach of the invention is advantageous as it avoids these problem(s).

According to the present invention an alternative to the iPS approach to generating one type of cell or tissue from another is transdifferentiation whereby a set of transcription factors and/or other factors such as small molecules are used to directly transform one type of cell into another.

Suitably the starting cell is not a totipotent cell.

Suitably the starting cell is not a pluripotent cell.

Suitably the starting cell is not an induced pluripotent stem cell (iPSC).

Suitably the starting cell is not an embryonic stem cell (ESC).

Suitably the starting cell is a mesenchymal stem cell (MSC).

Xu et al 2018 (Cell Biol. Toxicol. vol 34, pages 417-419 “CD8+iT cell, a budding star for cancer immunotherapy.”) disclose using haematopoietic stem cells and differentiating them directly to T-cells. The inventors submit that this is not the same as the invention, as HSCs do normally differentiate to T-cells. In more detail, Xu et al. disclose induction of Hoxb5 transcription factor in mouse B-cell progenitor cells. The Xu et al. method is carried out in vivo; suitably the method of the invention is carried out in vitro. Suitably the vertebrate somatic cell of the invention is not a B-cell. Suitably the vertebrate somatic cell of the invention is not a pro-pre-B-cell. Suitably the vertebrate somatic cell is not a B-cell progenitor. Suitably the vertebrate somatic cell is a human cell. In one embodiment suitably the transcription factor is not Hoxb5. In one embodiment, suitably the method of the invention does not involve Hoxb5. In one embodiment suitably Hoxb5 transcription factor is omitted. In one embodiment suitably the final or destination cell is not an early T-cell lineage progenitor like cell (ETP).

It is an advantage that induction of reporter activation in the Bcl11b-mCherry reporter in MSCs and MEFs is demonstrated by induction of the TET transcription factors (TET is an abbreviation and means “Tcf7+Ets1+Tbet”). Optimised results may be obtained by optionally also inducing expression of additional transcription factors as herein.

We show that combinations of TFs, using 17 TFs as an example, induce Bcl11b reporter activation in 2 different cell types—indication of a robust reprogramming (transdifferentiation) process—which is an advantage of the invention.

We show that T-cell reprogramming measured by Bcl11b-reporter activation is fast and asynchronous, which are further advantages.

We show that subsets of TFs contained within the exemplary combination of 9 TFs induce colonies that expand in culture. This shows minimal empirical sets of TFs which simplify the procedure, which is an advantage.

We demonstration that the transcription factor Tbet is a central TF for reprogramming (transdifferentiation according to the present invention) suggesting that CD8 T cell program or CD4 Th1 are induced.

We describe a minimal/empirical set of three TFs—Tbet, Ets1 and Tcf7—are effective for reprogramming (transdifferentiation according to the present invention) and Bcl11b-reporter activation.

Further particular and preferred aspects are set out in the accompanying independent and dependent claims. Features of the dependent claims may be combined with features of the independent claims as appropriate, and in combinations other than those explicitly set out in the claims.

Where an apparatus feature is described as being operable to provide a function, it will be appreciated that this includes an apparatus feature which provides that function or which is adapted or configured to provide that function.

### BRIEF DESCRIPTION OF THE DRAWINGS

Embodiments of the present invention will now be described further, with reference to the accompanying drawings, in which:

FIG. 1 shows a diagram. Cell states can be imagined as “gravity wells” or a series of valleys.
(a) (for comparison) a cell is moved from one stable differentiated state to a stable iPSC state and then back down a valley into a different differentiated state;
(b) (the invention) with transdifferentiation, a cell is moved directly from one differentiated state to another differentiated state.

FIG. 2 shows a bar chart. The bar chart shows transcription factor expression in MSCs derived from BM and dental pulp.

FIG. 3A shows photographs; FIG. 3B shows a bar chart.

FIG. 4A shows a diagram; FIG. 4B shows a bar chart; FIG. 4C shows a diagram.

FIG. 5A shows photographs; FIG. 5B shows a bar chart.

FIG. 6 shows a bar chart.

FIG. 7A shows a bar chart and FIG. 7B shows photographs.

FIG. 8 shows photographs.

FIG. 9 shows a bar chart.

FIG. 10 shows a bar chart.

FIG. 11 shows plots.

FIG. 12 shows plots.

FIG. 13 shows plots.

FIG. 14 shows a bar chart.

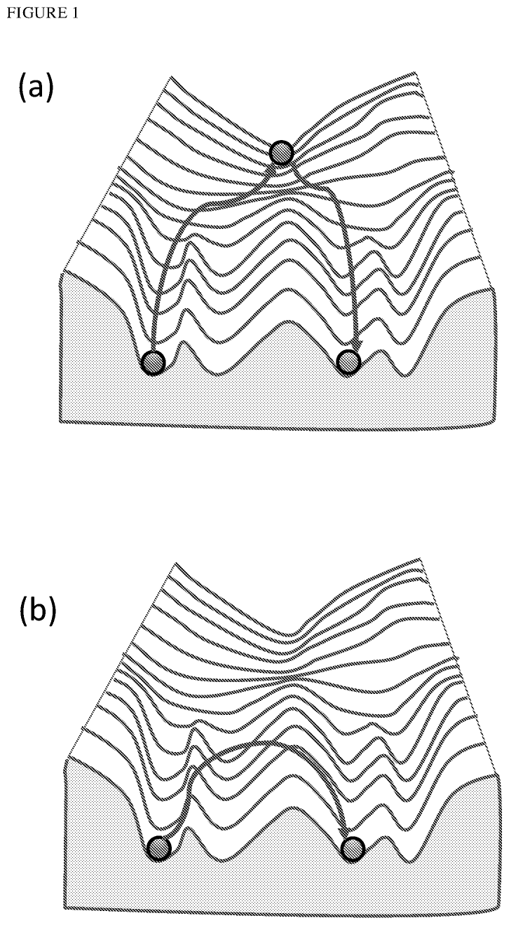

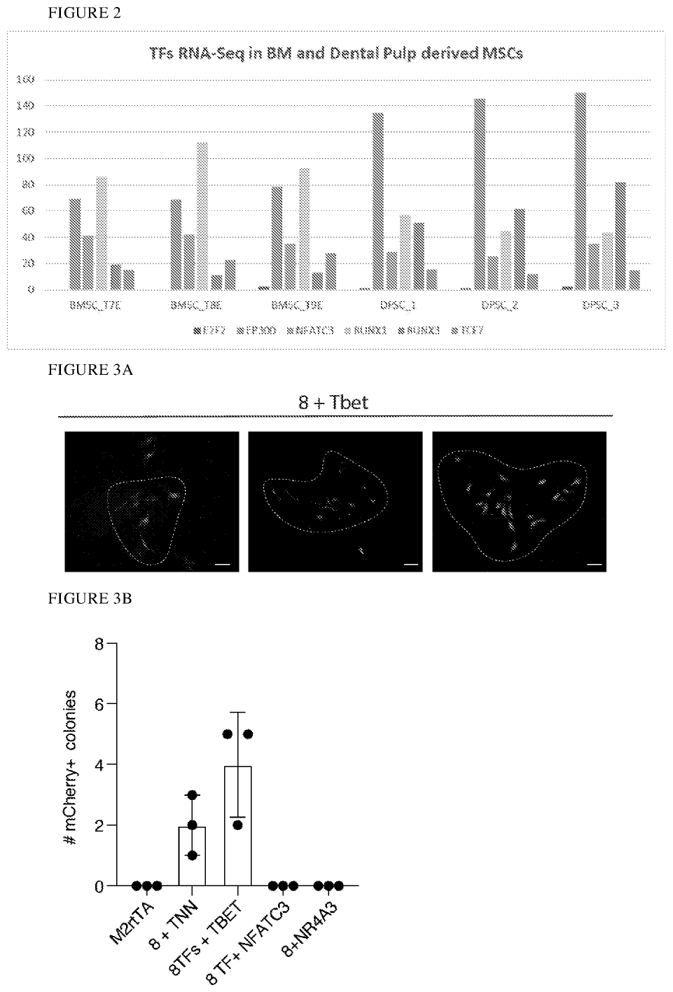

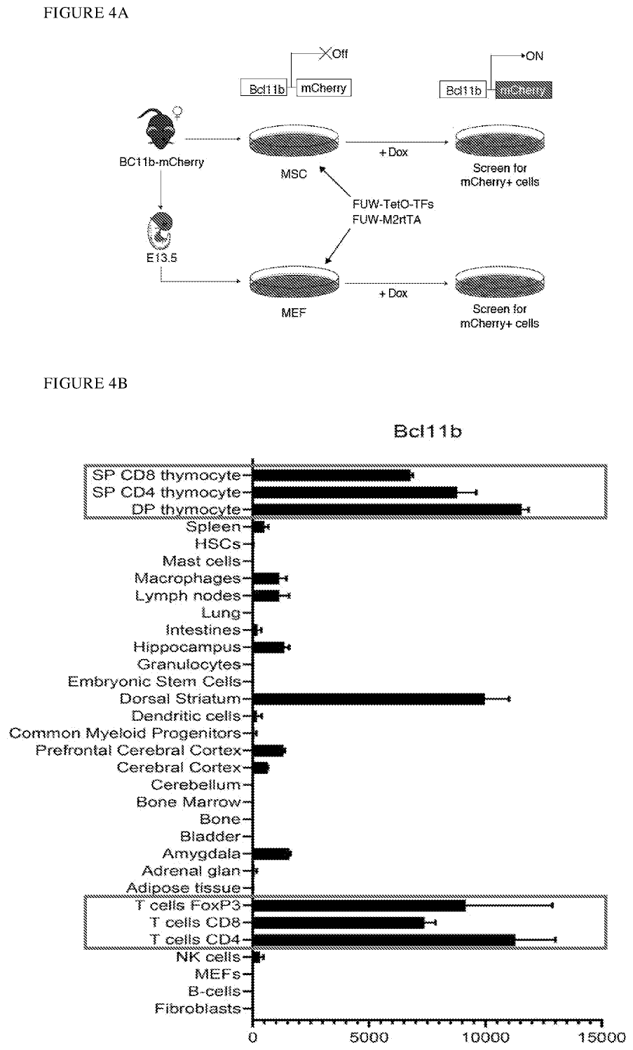

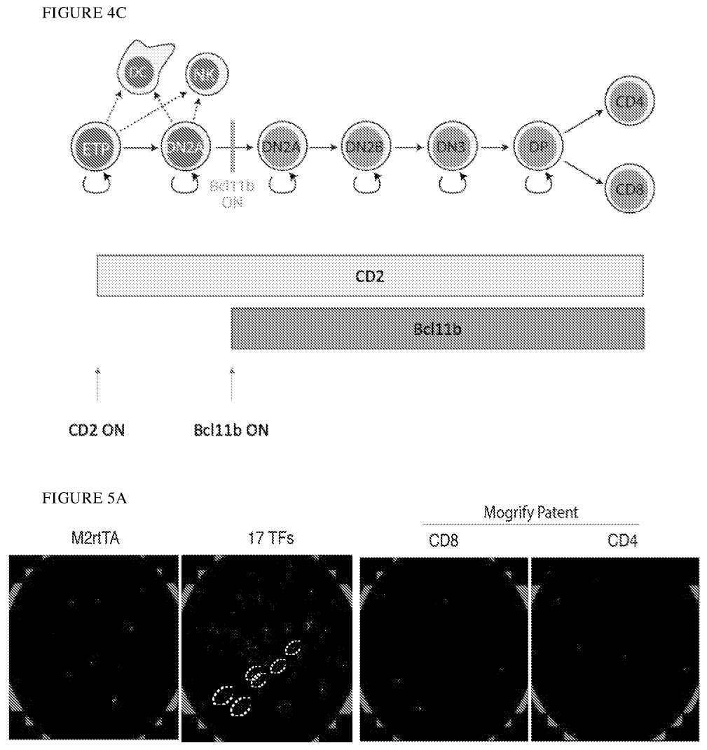

### EXAMPLES

### Example 1: Transdifferentiation

Here we demonstrate a method comprising the following steps (a) to (c):
(a) providing a vertebrate somatic cell

In this example the vertebrate somatic cell is a mesenchymal stem cell.

Suitably the mesenchymal stem cell is obtained from human bone marrow aspirate. An exemplary process is:
Harvest bone marrow cells from a normal donor by bone marrow aspiration.
Isolate mononuclear cells by density gradient isolation using Ficoll.
Isolated mononuclear cells are cultured on a plastic surface and cultured in DMEM with FBS or Human Platelet Lysate. Non adherent cells are washed off.
The adherent cells are the MSCs.
(b) inducing expression in said cell of at least three transcription factors, wherein said transcription factors are:
   i. Tbet (Tbx21),
   ii. Tcf7, and
   iii. Ets1.

In this example the at least three transcription factors consist of:
i. Tbet (Tbx21)
ii. Tcf7
iii. Ets1
iv. Bcl11b
v. Satb1
vi. Gata3
vii. Lef1
viii. Rorc; and
ix. Ikzf1.

Expression of these transcription factors is induced by electroporating the cell in the presence of a nucleic acid encoding the gene products of these transcription factors. In this example the nucleic acid is synthetic mRNA.

In this example the electroporation protocol is:
Cells are washed in PBS and re-suspended in electroporation medium (iso-osmolar, low salt buffer).
Synthetic mRNA is added to the cells at 0.4 ug/million cells.
Cell suspension/mRNA mix are transferred to an electroporation cuvette.
Electrical pulse is delivered to the cells.
Cells are immediately harvested and washed in complete medium and suspended in complete medium and returned to the incubator.
(c) and incubating said cell to allow transdifferentiation.

In this example, incubating is carried out after electroporation by placing the cell into complete medium (in this example complete medium=DMEM with 10% FCS+L-Glutamine) and incubating at 37° C. for 24-96 hours or until transdifferentiation takes place.

Medium is changed to RPMI after 3 days

Thus said cell is transdifferentiated to an such as to an immune effector cell, for example an effector T-cell.

### Example 2

Optional Genetic Modification Step:

A stock of bone-marrow derived MSCs is optionally transduced with a retroviral vector which expresses a CD19 CAR and the sort-suicide gene RQR8. MSCs expressing the CAR are purified using QBEND/10 beads which recognize RQR8. This is used as the starting cell (vertebrate somatic cell).

Main Method:

If not carrying out the optional genetic modification step, a stock of bone-marrow derived MSCs is used as the starting cell (vertebrate somatic cell).

In either case, next these (optionally transduced) MSCs are electroporated with synthetic mRNA encoding the transcription factors.

Electroporated MSCs are incubated in culture media RPMI supplemented with 5% FCS and L-Glutamine with IL7 and IL15.

They are fed with fresh media and split every two days.

When morphological changes are evident that suggest T-cell trans-differentiation (round, lymphocyte morphology), they are, or suitably a sample is, analysed by flow cytometry for T-cell markers such as CD2, CD4, CD5, CD7 and CD8.

Alternatively, or in addition, the cells may be analysed by flow cytometry for effector immune cell marker(s) such as CD3 (CD3e).

Suitably if a cell is CD2+CD5+CD7+ it is considered to be transdifferentiated to an effector immune cell.

Effector immune cells such as T-cells may emerge from this process and may be a mixture of CD4+CD8−, CD4-CD8+ and CD4+CD8+. Thus suitably cells are CD2+, CD5+ and CD7+ and also express either or both CD4 and CD8.

When sufficient T-cells are present in the culture, they are isolated by magnetic bead separation (e.g. Miltenyi CD4/CD8 beads).

In this example ‘sufficient’ depends on the needs of the skilled worker. Most suitably this may be considered as being a proportion of the cells in the culture; suitably sufficient means at least 10% of the cells in the culture are T-cells (i.e. cells displaying the marker or marker sets as outlined above).

These transdifferentiated T-cells (optionally CAR T-cells if optionally transduced with CAR as above) are now tested for function. If optionally transduced with CD19 CAR in the optional genetic modification step, this function may be tested against CD19+ targets using standard methods of cytotoxicity assays, cytokine release and proliferation.

### Example 3: 9 Transcription Factors Induce Bcl11b-Positive Colonies

Method

To identify inducing factors of T cell identity, 17 candidate TFs were expressed in primary cultures of mouse embryonic fibroblasts (MEFs) harbouring a T cell-specific reporter system (Bcl11b^{mCh(neo)/mCh(neo)} mouse, hereafter called Bcl11b-mCherry) (Ng et al. (2018) ‘A stochastic epigenetic switch controls the dynamics of T-cell lineage commitment.’ Elife, 7, e37851). Bcl11b is expressed specifically in the T cell lineage.

Isolation of MEFs (Mouse Embryonic Fibroblasts)

Homozygous Bcl11b-mCherry animals were crossed to generate homozygous Bcl11b-mCherry embryos. All animals were housed under controlled temperature (23±2° C.), subject to a fixed 12-h light/dark cycle, with free access to food and water. Primary cultures of MEFs were isolated at E13.5. Head, fetal liver and all internal organs were removed and the remaining tissue was mechanically dissociated. Dissected tissue was enzymatic digested using 0.12% trypsin/0.1 mM Ethylenediaminetetraacetic acid (EDTA) solution (3 mL per embryo), and incubation at 37° C. for 15 min. Additional 3 mL of same solution per embryo were added, followed by another 15 min incubation period. A single cell suspension was obtained and plated in 0.1% gelatin-coated 10-cm tissue culture dishes in growth media. Cells were grown for 2-3 days until confluence, dissociated with Tryple Express and frozen in Fetal Bovine Serum (FBS) 10% dimethyl sulfoxide (DMSO). Before plating for lentiviral transduction, MEFs were sorted to remove residual CD45⁺ and tdTomato⁺ cells that could represent cells with hematopoietic potential. MEFs used for screening and in the following experiments were tdTomato⁻ CD45⁻ with purity above 99% and expanded up to 4 passages.

Transduction

Bcl11b-mCherry MEFs were maintained in growth medium [Dulbecco's modified eagle medium (DMEM) supplemented with 10% (v/v) FBS, 2 mM L-Glutamine and antibiotics (10 μg/ml Penicillin and Streptomycin)]. All cells were maintained at 37° C. and 5% (v/v) CO2. Bcl11b-mCherry MEFs were seeded at a density of 40,000 cells per well on 0.1% gelatin coated 6-well plates. Cells were incubated overnight with a ratio of 1:1 FUW-TetO-TFs and FUW-M2rtTA lentiviral particles in growth media supplemented with 8 μg/mL polybrene. Cells were transduced twice in consecutive days and after overnight incubation, media was replaced with fresh growth media. After the second transduction, growth media was supplemented with Doxycycline (1 μg/mL)—day 0. Media was changed every 2-3 days for the duration of the cultures. Emerging mCherry positive cells were analyzed 3-12 days post-transduction by microscopy. The entire well of a 6-well plate was acquired in an automated Zeiss CD7.

Flow Cytometry

To assess CD3 expression at cell surface, transduced Bcl11b-mCherry MEFs were dissociated with TrypLE Express, resuspended in PBS 2% (v/v) FBS and incubated with anti-mouse CD3 (Biolegend, 100220) for 30 min on ice and protected from light. To prevent unspecific binding, rat serum was used in a 1:100 dilution. After incubation, cells were washed and resuspended with PBS 2% FBS. DAPI staining was performed at a concentration of 1 μg/mL to exclude dead cells. Samples were analysed in a BD LSR II. Data analysis was performed using FlowJo Software (version 10.7, FlowJo, LLC).

We refer to FIG. 3 which shows that expression of the 9 transcription factors induces Bcl11b-positive colonies.

The colony assays are based on the number of Bcl11b-reporter positive colonies.

In more detail, FIG. 3 shows that enforced expression of 9 transcription factors activates a T-cell specific reporter. Quantification of Bcl11b-mCherry positive colonies 9 days after adding DOX is shown. MEFS were transduced with M2rtTA only or co-transduced with M2rtTA and a combination of 11 TFs (Bcl11b, Lef1, Tcf7, Ets1, Gata3, Ikzf1, Satb1, Rorc, Tbet, Nr4a3, Nfatc3). Additionally, M2rtTA was co-transduced with 8 TFs pool (Bcl11b, Lef1, Tcf7, Ets1, Gata3, Ikzf1, Satb1, Rorc) plus one TF from the following three: Tbet, Nr4a3, Nfatc3. Mean+SD of 3 replicates is shown.

Fluorescence Microscopy pictures of mCherry positive colonies generated with 9 TFs (Bcl11b, Lef1, Tcf7, Ets1, Gata3, Ikzf1, Satb1, Rorc, Tbet), 9 days after adding Dox. Scale bar=200 μm.

### Example 4: Comparative Data

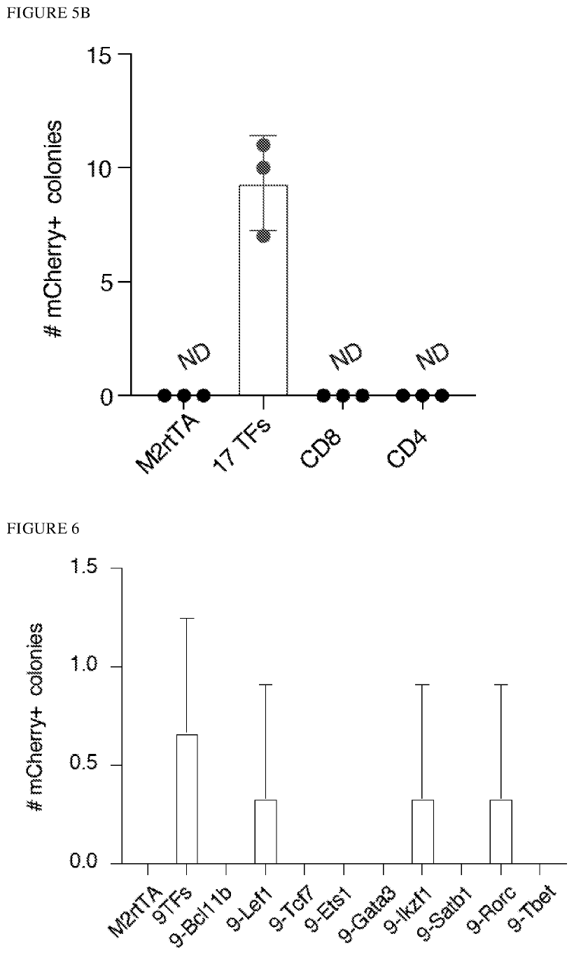

Mogrify Limited, Company no. 10002103, 19 Aberdeen Avenue, Cambridge, CB2 8DL, United Kingdom offer tools to determine which transcription factors can programme 35 which cell fates/cell lineages. FIG. 5 shows how the Mogrify predicted transcription factor combinations of the prior art do not work. By comparison a transcription factor combination according to the present invention does work.

In particular in FIG. 5A compare photographs 1 (M2rtTA), 3 (CD8) and 4 (CD4) (not the invention) with photograph 2 (17 TFs) (embodiment of the invention).

In particular in FIG. 5B compare bars 1 (M2rtTA), 3 (CD8) and 4 (CD4) (not the invention) with bar 2 (17 TFs) (embodiment of the invention).

“17TFs” in this example means induction of Bcl11b, Lef1, Tcf7, Stat4, Tox, Ets1, Gata3, Tbx21, Nr4a3, Ikzf1, Nfatc3, Satb1, Rorc, Zbtb16, Nfil3, Znf683 and Tcf12.

Mogrify CD4+ in this example means induction of RorA, Jun, Fos, Lef1 and Bach2.

Mogrify CD8+ in this example means induction of RorA, Jun, Fos, Smad7 and Runx3.

### Example 5

The inventors worked to optimise towards an empirical set. Here we show using a subtractive approach that LEF1, IKZF1 and RORc are dispensable for Bcl11b-reporter activation.

We refer to FIG. 6. This shows Bcl11b-mCherry MEFs, 1 experiment with 3 replicates, Mean±SD.

“9 TFs”: induction of Bcl11b, Lef1, Tcf7, Ets1, Gata3, Ikzf1, Satb1, Rorc and Tbet. “9-[X]”: induction of 8 TFs comprising the above 9 TFs, without the recited TF. So “9-Lef1” means induction of Bcl11b, Tcf7, Ets1, Gata3, Ikzf1, Satb1, Rorc and Tbet (i.e. not Lef1).

In more detail, FIG. 6 shows that LEF1, IKZF1 and RORc are dispensable for Bcl11b-reporter activation. Quantification of Bcl11b-mCherry positive colonies per TF combination at day 9 of reprogramming. MEFs were transduced with control M2rtTA or co-transduced with M2rtTA and 9 TFs (Bcl11b, Lef1, Tcf7, Ets1, Gata3, Ikzf1, Satb1, Rorc, Tbet). Alternatively, combinations where one transcription factor was individually removed from the 9 TF pool were used to define essential TFs for Bcl11b-mCherry reporter activation. Mean+SD of 3 replicates is shown.

These results demonstrate that removal of LEF1, IKZF1 and RORc from TF combination do not inhibit Bcl11b-mCherry reporter activation, demonstrating that these TFs are not required for reporter activation.

### Example 6

The inventors worked to optimise towards an empirical set. Here we show using a complex pooling approach that TBET, TCF7 and ETS1 are required to activate the Bcl11b-reporter.

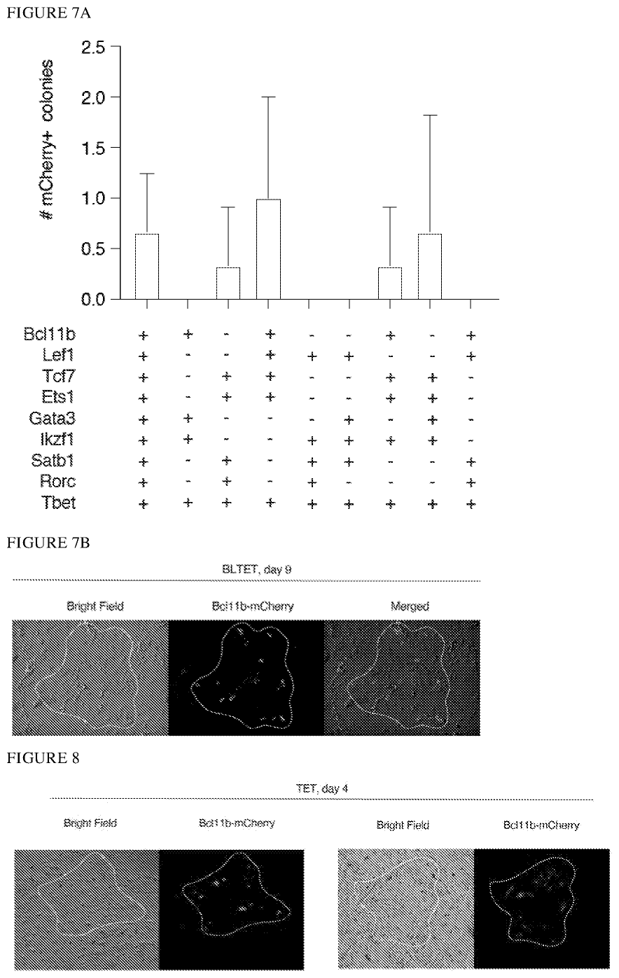

We refer to FIG. 7. This shows Bcl11b-mCherry MEFs, 1 experiment with 3 replicates, Mean±SD.

FIG. 7A shows a bar chart which reveals active combinations of TFs.

FIG. 7B shows Bcl11b reporter assay for the ‘TET’ group of three TFs (i.e. induction of TBET, TCF7 and ETS1).

In more detail, FIG. 7 shows that TBET, TCF7 and ETS1 are required to activate the Bcl11b-reporter. Quantification of Bcl11b-mCherry positive colonies per TF combination 9 days after adding DOX. MEFs were co-transduced with M2rtTA and 9 TFs (Bcl11b, Lef1, Tcf7, Ets1, Gata3, Ikzf1, Satb1, Rorc, Tbet) or small pools of 4 or 5 TFs from the 9 TFs list. Mean+SD of 3 replicates is shown. Fluorescence Microscopy pictures of mCherry positive colonies generated with 5 TFs (Bcl11b, Lef1, Tcf7, Ets1, Tbet), 9 days after adding Dox.

These results show that removal of TBET, TCF7 and ETS1 from the pool of 9 TFs abolish Bcl11b-mCherry reporter activation, suggesting that these TFs are needed to induce T-cells.

This result indicates that TBET, TCF7 and ETS1 may the minimal regulatory network controlling T cell induction. In other words we show that ‘TET’ induction (i.e. induction of TBET, TCF7 and ETS1) is effective for T cell induction.

Thus the ‘TET’ group of three TFs (i.e. induction of TBET, TCF7 and ETS1) may be the minimal empirical set of TFs needed for T cell induction.

### Example 7

In this example we show further evidence that induction of the empirical set of three TFs (TBET, ETS1 and TCF7) generates Bcl11b-reporter positive colonies.

We refer to FIG. 8. This shows Bcl11b-mCherry MEFs, 1 experiment with 3 replicates. This result is clear at day 4 which shows an advantageous timescale for transdifferentiation according to the present invention.

### Example 8—Further Exemplary Sets of TFs

Additional factors may be advantageous for optimal efficiency/colony expansion.

Here we demonstrate exemplary combinations of TFs.

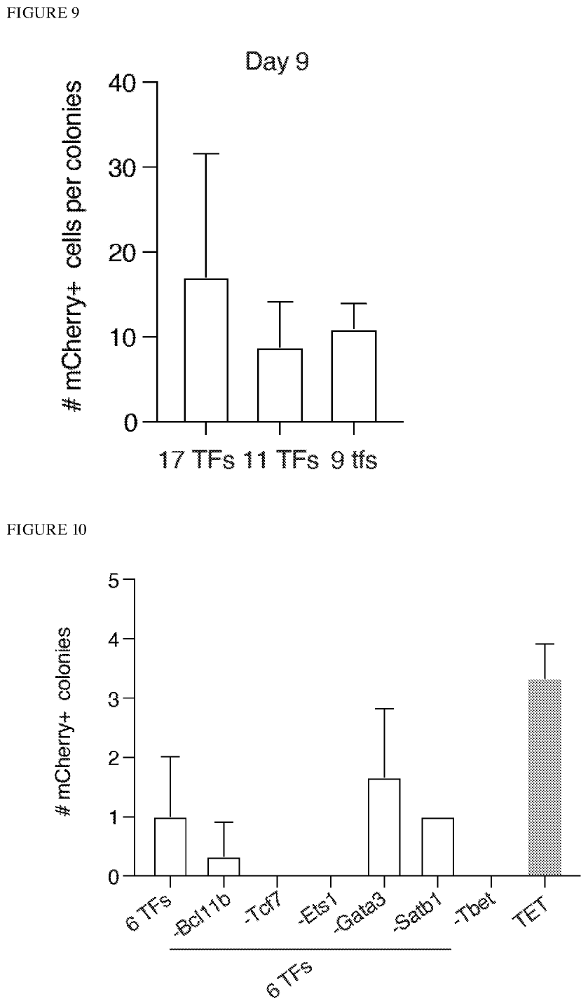

We refer to FIG. 9. This shows Bcl11b-mCherry MEFs, 1 experiment with 3 replicates.
“17 TFs” means induction of expression of: Bcl11b, Lef1, Tcf7, Stat4, Tox, Ets1, Gata3, Tbx21, Nr4a3, Ikzf1, Nfatc3, Satb1, Rorc, Zbtb16, Nfil3, Znf683, Tcf12.
“11 TFs” means induction of expression of: Bcl11b, Lef1, Tcf7, Ets1, Gata3, Ikzf1, Satb1, Rorc, Tbet, Nfatc3, Nr4a3.
“9 TFs” means induction of expression of: Bcl11b, Lef1, Tcf7, Ets1, Gata3, Ikzf1, Satb1, Rorc, Tbet.

### Example 9—Exemplary TET Combination

Induction of Tbet, Tcf7 and Ets1 in combination (i.e. the ‘TET group’ described herein—i.e. TCF1/TCF7, TBET and ETS1) activates the Bcl11b-reporter.

We provide confirmation that the Tbet, Tcf7 and Ets1 combination is sufficient to activate Bcl11b-reporter. However, if any of the three is removed, no activation takes place.

We refer to FIG. 10—Bcl11b-mCherry MEFs—1 independent experiment with 3 replicates—Mean±SD.

In more detail, FIG. 10 shows that TBET, TCF7 and ETS1 are sufficient to activate Bcl11b-reporter. Quantification of Bcl11b-mCherry positive colonies 9 days after adding DOX. MEFS were transduced with M2rtTA only or co-transduced with M2rtTA and a combination of 6 TFs (Bcl11b, Tcf7, Ets1, Gata3, Satb1, Tbet). Additional combinations are shown where one transcription factor was individually excluded from the 6 TF pool or the expression of TBET, TCF7 and ETS1 (3 TFs). Mean+SD of 3 replicates is shown. Fluorescence Microscopy pictures of tdTomato positive colonies generated with TBET, ETS1, TCF7, 4 and 7 days after adding Dox.

### Example 10—TET Combination Induces CD3

Induction of Tbet, Tcf7 and Ets1 in combination (i.e. the ‘TET group’ described herein—i.e. TCF1/TCF7, TBET and ETS1) induces CD3 positive cells.

We refer to FIG. 11—Bcl11b-mCherry MEFs, day 15. Staining with CD3 shows a clear population (4.36%) of CD3+ cells in the cells expressing the Tbet, Tcf7 and Ets1 combination.

In more detail, we provide evidence that TBET, TCF7 and ETS1 in combination induces CD3 at cell surface. Figure n1 shows flow cytometry plots for Bcl11b-mCherry and CD3 expression on mouse embryonic fibroblasts (MEFs) co-transduced with M2rtTA, TBET, ETS1 and TCF7, or transduced with M2rtTA as a control 15 days after adding DOX.

FIG. 11 shows enforced expression of TBET, ETS1 and TCF7 in mouse fibroblasts generate CD3-positive cells. Representative flow cytometry plots of mouse embryonic fibroblasts (MEFs), 15 days after transduction with the 3 transcription factors individually cloned into Dox-inducible FUW-TetO lentiviral vectors. FUW-M2rtTA (plasmid number #20342 from ADDGENE (ibid.)) was used as control.

### Example 11—TET Combination Induces CD3 and CD45

Expression of CD3 is taken as a marker of transdifferentiation to an immune effector cell.

Expression of CD45 is taken as a marker of transdifferentiation to an immune effector cell.

Here we show that induction of Tbet, Ets1 and Tcf7 (TET combination) in a vertebrate somatic cell (in this example human cells—HEFs) results in transdifferentiation to an immune effector cell.

In other words we demonstrate that the TET combination is sufficient to generate human CD45+ cells and CD3+ cells from vertebrate somatic cells.

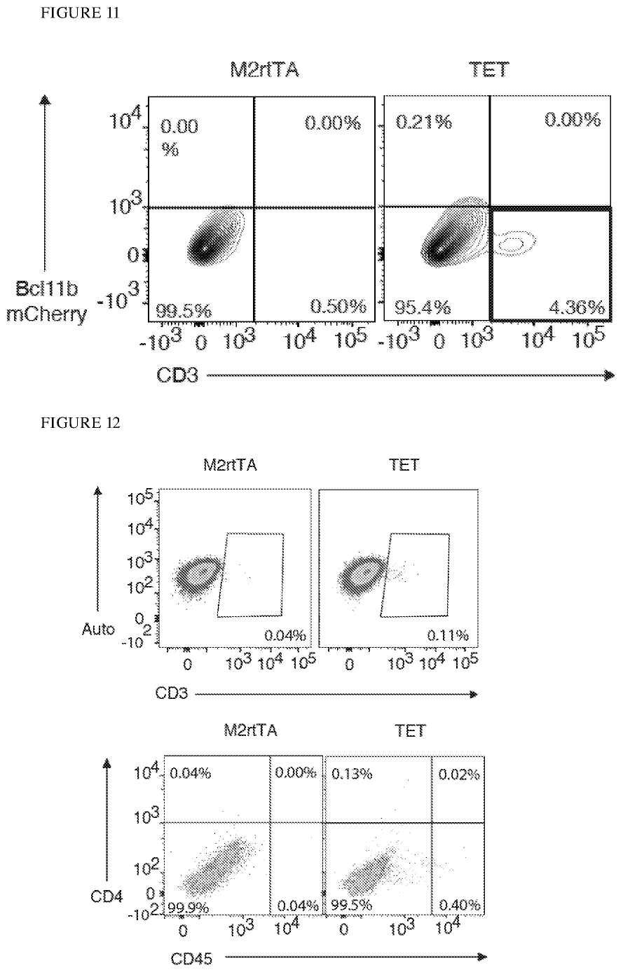

We refer to FIG. 12, which shows induction of CD3+(upper) and CD45+(lower) populations at low frequency populations.

FIG. 12 shows TBET, ETS1 and TCF7 induce CD3 and CD45 expression in Human fibroblasts. Representative flow cytometry plots of human embryonic fibroblasts (HEFs), 12 days (top) and 15 days (bottom) after transduction with TBET, ETS1 and TCF7 cloned into Dox-inducible FUW-TetO lentiviral vectors. FUW-M2rtTA was used as control.

### Example 12—Polycistronic Construct of Tbet, Ets1 and Tcf7 Increase CD8+ Population

We show that polycistronic constructs inducing the expression of several transcription factors from a single nucleic acid are effective in producing transdifferentiation to an immune effector cell. In this example expression of CD8 (CD8+) shows the cell has become an immune effector cell.

In particular we show that in a polycistronic construct, the order Tbet-Ets1-Tcf7 works the best giving around 11% CD8+ cells.

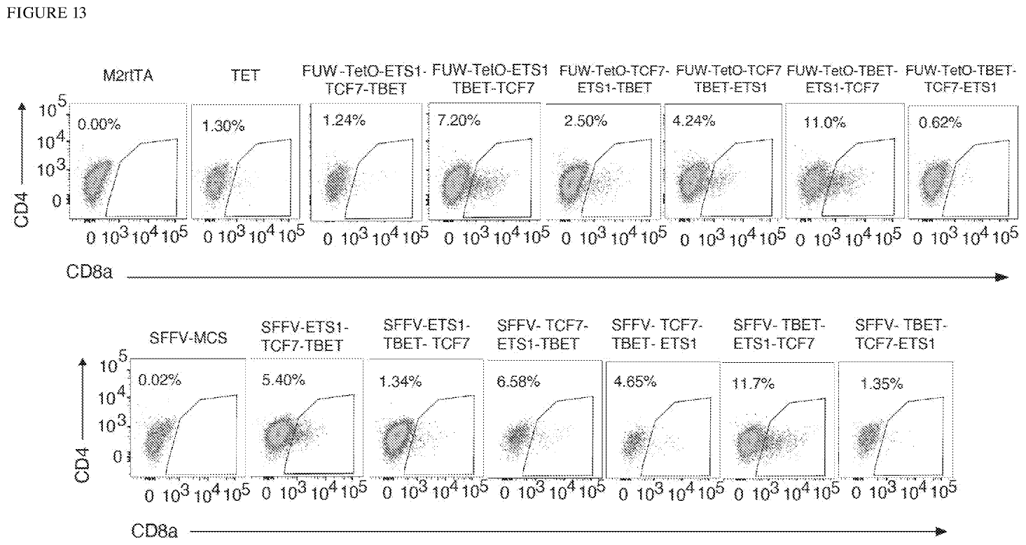

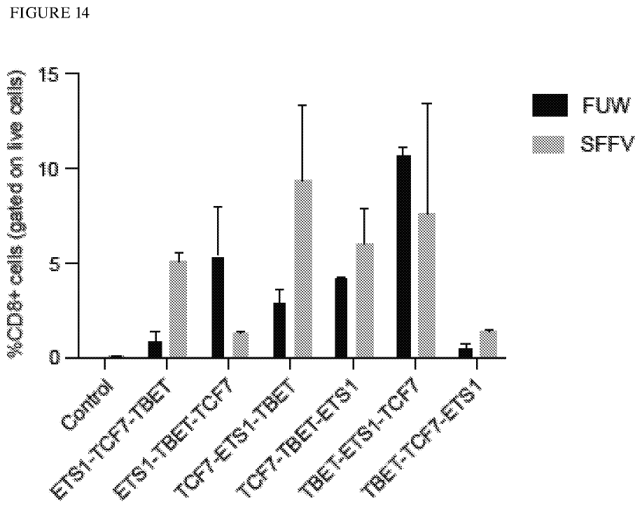

We refer to FIGS. 13 and 14. Vertebrate somatic cells (in this example human cells—HEFs) were used. Data collected at day 8.

FIG. 13 shows polycistronic constructs for TBET, ETS1 and TCF7 induce CD8 expression in Human fibroblasts. Schematic representation of polycistronic constructions (Top). Representative flow cytometry plots of human embryonic fibroblasts (HEFs), 9 days after transduction with polycistronic constructs encoding TBET, ETS1 and TCF7 cloned into Dox-inducible FUW-TetO (middle) or constitutive promoter SFFV (bottom) lentiviral vectors. Transduction with a pool of TBET, ETS1 and TCF7 cloned individually into FUW-TetO (TET) was included for comparison. FUW-M2rtTA and SFFV-multiple cloning site (MCS) were used as control.

FIG. 14 shows TBET-ETS1-TCF7 polycistronic vector induce robust CD8 expression. Human embryonic fibroblasts were transduced with polycistronic constructs encoding TBET, ETS1 and TCF7 in the displayed order. Dox-inducible FUW-TetO (FUW) or constitutive promoter SFFV (SFFV) lentiviral vectors were used. The graph shows the percentage of CD8+ cells induced with each polycistronic vector 9 days after transduction (Mean+SD, n=2).

Although illustrative embodiments of the invention have been disclosed in detail herein, with reference to the accompanying drawings, it is understood that the invention is not limited to the precise embodiment and that various changes and modifications can be effected therein by one skilled in the art without departing from the scope of the invention as defined by the appended claims and their equivalents.

Exemplary Sequences
SFFV-TBET-ETS1-TCF7
expression cassette
Coding sequence in UPPER CASE
Ancillary sequences (incl. promoter)
in lower case
SEQ ID NO: 5
gtaacgccattttgcaaggcatggaaaaataccaaaccaagaata
gagaagttcagatcaagggcgggtacatgaaaatagctaacgttg
ggccaaacaggatatctgcggtgagcagtttcggccccggcccgg
ggccaagaacagatggtcaccgcagtttcggccccggcccgaggc
caagaacagatggtccccagatatggcccaaccctcagcagtttc
ttaagacccatcagatgtttccaggctcccccaaggacctgaaat
gaccctgcgccttatttgaattaaccaatcagcctgcttctcgct
tctgttcgcgcgcttctgcttcccgagctctataaaagagctcac
aacccctcactcggcgcgccagtcctccgacagactgagtcggcc
ggtcgaatcaagcttatcgataccgtcgacggatccggatccgAT
GGGCATCGTGGAGCCGGGTTGCGGAGACATGCTGACGGGCACCGA
GCCGATGCCGGGGAGCGACGAGGGCCGGGCGCCTGGCGCCGACCC
GCAGCACCGCTACTTCTACCCGGAGCCGGGCGCGCAGGACGCGGA
CGAGCGTCGCGGGGGCGGCAGCCTGGGGTCTCCCTACCCGGGGGG
CGCCTTGGTGCCCGCCCCGCCGAGCCGCTTCCTTGGAGCCTACGC
CTACCCGCCGCGACCCCAGGCGGCCGGCTTCCCCGGCGCGGGCGA
GTCCTTCCCGCCGCCCGCGGACGCCGAGGGCTACCAGCCGGGCGA
GGGCTACGCCGCCCCGGACCCGCGCGCCGGGCTCTACCCGGGGCC
GCGTGAGGACTACGCGCTACCCGCGGGACTGGAGGTGTCGGGGAA
ACTGAGGGTCGCGCTCAACAACCACCTGTTGTGGTCCAAGTTTAA
TCAGCACCAGACAGAGATGATCATCACCAAGCAGGGACGGCGGAT
GTTCCCATTCCTGTCATTTACTGTGGCCGGGCTGGAGCCCACCAG
CCACTACAGGATGTTTGTGGACGTGGTCTTGGTGGACCAGCACCA
CTGGCGGTACCAGAGCGGCAAGTGGGTGCAGTGTGGAAAGGCCGA
GGGCAGCATGCCAGGAAACCGCCTGTACGTCCACCCGGACTCCCC
CAACACAGGAGCGCACTGGATGCGCCAGGAAGTTTCATTTGGGAA
ACTAAAGCTCACAAACAACAAGGGGGCGTCCAACAATGTGACCCA
GATGATTGTGCTCCAGTCCCTCCATAAGTACCAGCCCCGGCTGCA
TATCGTTGAGGTGAACGACGGAGAGCCAGAGGCAGCCTGCAACGC
TTCCAACACGCATATCTTTACTTTCCAAGAAACCCAGTTCATTGC
CGTGACTGCCTACCAGAATGCCGAGATTACTCAGCTGAAAATTGA
TAATAACCCCTTTGCCAAAGGATTCCGGGAGAACTTTGAGTCCAT
GTACACATCTGTTGACACCAGCATCCCCTCCCCGCCTGGACCCAA
CTGTCAATTCCTTGGGGGAGATCACTACTCTCCTCTCCTACCCAA
CCAGTATCCTGTTCCCAGCCGCTTCTACCCCGACCTTCCTGGCCA
GGCGAAGGATGTGGTTCCCCAGGCTTACTGGCTGGGGGCCCCCCG
GGACCACAGCTATGAGGCTGAGTTTCGAGCAGTCAGCATGAAGCC
TGCATTCTTGCCCTCTGCCCCTGGGCCCACCATGTCCTACTACCG
AGGCCAGGAGGTCCTGGCACCTGGAGCTGGCTGGCCTGTGGCACC
CCAGTACCCTCCCAAGATGGGCCCGGCCAGCTGGTTCCGCCCTAT
GCGGACTCTGCCCATGGAACCCGGCCCTGGAGGCTCAGAGGGACG
GGGACCAGAGGACCAGGGTCCCCCCTTGGTGTGGACTGAGATTGC
CCCCATCCGGCCGGAATCCAGTGATTCAGGACTGGGCGAAGGAGA
CTCTAAGAGGAGGCGCGTGTCCCCCTATCCTTCCAGTGGTGACAG
CTCCTCCCCTGCTGGGGCCCCTTCTCCTTTTGATAAGGAAGCTGA
AGGACAGTTTTATAACTATTTTCCCAACGGCAGCGGCGCCACAAA
CTTCTCTCTGCTAAAGCAAGCAGGTGATGTTGAAGAAAACCCCGG
GCCTATGAGCTACTTTGTGGATTCTGCTGGGAGCAGCCCCGTCCC
TTACTCAGCGCCTCGTCCTGCAGTGGTGAGGCAAGGACCTAGCAA
CACTTATGAAGATCCTCGAATGAACTGTGGTTTCCAGTCCAATTA
TCACCAGCAAAGACCTTGCTACCCCTTTTGGGATGAGATGGCAAC
TCAGGAAGTTCCTACTGGTCTTGAACACTGTGTCTCAGATATGGA
ATGTGCAGATGTCCCACTATTAACTCCAAGCAGCAAAGAAATGAT
GTCTCAAGCATTAAAAGCTACTTTCAGTGGTTTCACTAAAGAACA
GCAACGACTGGGGATCCCAAAAGACCCCCGGCAGTGGACAGAAAC
CCATGTTCGGGACTGGGTGATGTGGGCTGTGAATGAATTCAGCCT
GAAAGGTGTAGACTTCCAGAAGTTCTGTATGAATGGAGCAGCCCT
CTGCGCCCTGGGTAAAGACTGCTTTCTCGAGCTGGCCCCAGACTT
TGTTGGGGACATCTTATGGGAACATCTAGAGATCCTGCAGAAAGA
GGATGTGAAACCATATCAAGTTAATGGAGTCAACCCAGCCTATCC
AGAATCCCGCTATACCTCGGATTACTTCATTAGCTATGGTATTGA
GCATGCCCAGTGTGTTCCACCATCGGAGTTCTCAGAGCCCAGCTT
CATCACAGAGTCCTATCAGACGCTCCATCCCATCAGCTCGGAAGA
GCTCCTCTCCCTCAAGTATGAGAATGACTACCCCTCGGTCATTCT
CCGAGACCCTCTCCAGACAGACACCTTGCAGAATGACTACTTTGC
TATCAAACAAGAAGTCGTCACCCCAGACAACATGTGCATGGGGAG
GACCAGTCGTGGTAAACTCGGGGGCCAGGACTCTTTTGAAAGCAT
AGAGAGCTACGATAGTTGTGATCGCCTCACCCAGTCCTGGAGCAG
CCAGTCATCTTTCAACAGCCTGCAGCGTGTTCCCTCCTATGACAG
CTTCGACTCAGAGGACTATCCGGCTGCCCTGCCCAACCACAAGCC
CAAGGGCACCTTCAAGGACTATGTGCGGGACCGTGCTGACCTCAA
TAAGGACAAGCCTGTCATTCCTGCTGCTGCCCTAGCTGGCTACAC
AGGCAGTGGACCAATCCAGCTATGGCAGTTTCTTCTGGAATTACT
CACTGATAAATCCTGTCAGTCTTTTATCAGCTGGACAGGAGATGG
CTGGGAATTCAAACTTTCTGACCCAGATGAGGTGGCCAGGAGATG
GGGAAAGAGGAAAAACAAACCTAAGATGAATTATGAGAAACTGAG
CCGTGGCCTACGCTACTATTACGACAAAAACATCATCCACAAGAC
AGCGGGGAAACGCTACGTGTACCGCTTTGTGTGTGACCTGCAGAG
CCTGCTGGGGTACACCCCTGAGGAGCTGCACGCCATGCTGGACGT
CAAGCCAGATGCCGACGAGGGCTCCGGCGAGGGCAGGGGAAGTCT
TCTAACATGCGGGGACGTGGAGGAAAATCCCGGCCCAATGCCGCA
GCTGGACTCCGGCGGGGGCGGCGCGGGCGGCGGCGACGACCTCGG
CGCGCCGGACGAGCTGCTGGCCTTCCAGGATGAAGGCGAGGAGCA
GGACGACAAGAGCCGCGACAGCGCCGCCGGTCCCGAGCGCGACCT
GGCCGAGCTCAAGTCGTCGCTCGTGAACGAGTCCGAGGGCGCGGC
CGGCGGCGCAGGGATCCCGGGGGTCCCGGGGGCCGGCGCCGGGGC
CCGCGGCGAGGCCGAGGCTCTCGGGCGGGAACACGCTGCGCAGAG
ACTCTTCCCGGACAAACTTCCAGAGCCCCTGGAGGACGGCCTGAA
GGCCCCGGAGTGCACCAGCGGCATGTACAAAGAGACCGTCTACTC
CGCCTTCAATCTGCTCATGCATTACCCACCCCCCTCGGGAGCAGG
GCAGCACCCCCAGCCGCAGCCCCCGCTGCACAAGGCCAATCAGCC
CCCCCACGGTGTCCCCCAACTCTCTCTCTACGAACATTTCAACAG
CCCACATCCCACCCCTGCACCTGCGGACATCAGCCAGAAGCAAGT
TCACAGGCCTCTGCAGACCCCTGACCTCTCTGGCTTCTACTCCCT
GACCTCAGGCAGCATGGGGCAGCTCCCCCACACTGTGAGCTGGTT
CACCCACCCATCCTTGATGCTAGGTTCTGGTGTACCTGGTCACCC
AGCAGCCATCCCCCACCCGGCCATTGTGCCCCCCTCAGGGAAGCA
GGAGCTGCAGCCCTTCGACCGCAACCTGAAGACACAAGCAGAGTC
CAAGGCAGAGAAGGAGGCCAAGAAGCCAACCATCAAGAAGCCCCT
CAATGCCTTCATGCTGTACATGAAGGAGATGAGAGCCAAGGTCAT
TGCAGAGTGCACACTTAAGGAGAGCGCTGCCATCAACCAGATCCT
GGGCCGCAGGTGGCACGCGCTGTCGCGAGAAGAGCAGGCCAAGTA
CTATGAGCTGGCCCGCAAGGAGAGGCAGCTGCACATGCAGCTATA
CCCAGGCTGGTCAGCGCGGGACAACTACGGGAAGAAGAAGAGGCG
GTCGAGGGAAAAGCACCAAGAATCCACCACAGGAGGAAAAAGAAA
TGCATTCGGTACTTACCCGGAGAAGGCCGCTGCCCCAGCCCCGTT
CCTTCCGATGACAGTGCTCTAG
Fuw-TetO-TBET-ETS1-TCF7 expression cassette
Coding sequence in UPPER CASE
Ancillary sequences (incl. promoter)
in lower case
SEQ ID NO: 6
tccctatcagtgatagagaaaagtgaaagtcgagtttaccactcc
ctatcagtgatagagaaaagtgaaagtcgagtttaccactcccta
tcagtgatagagaaaagtgaaagtcgagtttaccactccctatca
gtgatagagaaaagtgaaagtcgagtttaccactccctatcagtg
atagagaaaagtgaaagtcgagtttaccactccctatcagtgata
gagaaaagtgaaagtcgagtttaccactccctatcagtgatagag
aaaagtgaaagtcgagctcggtacccgggtcgagtaggcgtgtac
ggtgggaggcctatataagcagagctcgtttagtgaaccgtcaga
tcgcctggagacgccatccacgctgttttgacctccatagaagac
accgggaccgatccagcctccgcggccccgaattcaccggttcta
gagctagcATGGGCATCGTGGAGCCGGGTTGCGGAGACATGCTGA
CGGGCACCGAGCCGATGCCGGGGAGCGACGAGGGCCGGGCGCCTG
GCGCCGACCCGCAGCACCGCTACTTCTACCCGGAGCCGGGCGCGC
AGGACGCGGACGAGCGTCGCGGGGGCGGCAGCCTGGGGTCTCCCT
ACCCGGGGGGCGCCTTGGTGCCCGCCCCGCCGAGCCGCTTCCTTG
GAGCCTACGCCTACCCGCCGCGACCCCAGGCGGCCGGCTTCCCCG
GCGCGGGCGAGTCCTTCCCGCCGCCCGCGGACGCCGAGGGCTACC
AGCCGGGCGAGGGCTACGCCGCCCCGGACCCGCGCGCCGGGCTCT
ACCCGGGGCCGCGTGAGGACTACGCGCTACCCGCGGGACTGGAGG
TGTCGGGGAAACTGAGGGTCGCGCTCAACAACCACCTGTTGTGGT
CCAAGTTTAATCAGCACCAGACAGAGATGATCATCACCAAGCAGG
GACGGCGGATGTTCCCATTCCTGTCATTTACTGTGGCCGGGCTGG
AGCCCACCAGCCACTACAGGATGTTTGTGGACGTGGTCTTGGTGG
ACCAGCACCACTGGCGGTACCAGAGCGGCAAGTGGGTGCAGTGTG
GAAAGGCCGAGGGCAGCATGCCAGGAAACCGCCTGTACGTCCACC
CGGACTCCCCCAACACAGGAGCGCACTGGATGCGCCAGGAAGTTT
CATTTGGGAAACTAAAGCTCACAAACAACAAGGGGGCGTCCAACA
ATGTGACCCAGATGATTGTGCTCCAGTCCCTCCATAAGTACCAGC
CCCGGCTGCATATCGTTGAGGTGAACGACGGAGAGCCAGAGGCAG
CCTGCAACGCTTCCAACACGCATATCTTTACTTTCCAAGAAACCC
AGTTCATTGCCGTGACTGCCTACCAGAATGCCGAGATTACTCAGC
TGAAAATTGATAATAACCCCTTTGCCAAAGGATTCCGGGAGAACT
TTGAGTCCATGTACACATCTGTTGACACCAGCATCCCCTCCCCGC
CTGGACCCAACTGTCAATTCCTTGGGGGAGATCACTACTCTCCTC
TCCTACCCAACCAGTATCCTGTTCCCAGCCGCTTCTACCCCGACC
TTCCTGGCCAGGCGAAGGATGTGGTTCCCCAGGCTTACTGGCTGG
GGGCCCCCCGGGACCACAGCTATGAGGCTGAGTTTCGAGCAGTCA
GCATGAAGCCTGCATTCTTGCCCTCTGCCCCTGGGCCCACCATGT
CCTACTACCGAGGCCAGGAGGTCCTGGCACCTGGAGCTGGCTGGC
CTGTGGCACCCCAGTACCCTCCCAAGATGGGCCCGGCCAGCTGGT
TCCGCCCTATGCGGACTCTGCCCATGGAACCCGGCCCTGGAGGCT
CAGAGGGACGGGGACCAGAGGACCAGGGTCCCCCCTTGGTGTGGA
CTGAGATTGCCCCCATCCGGCCGGAATCCAGTGATTCAGGACTGG
GCGAAGGAGACTCTAAGAGGAGGCGCGTGTCCCCCTATCCTTCCA
GTGGTGACAGCTCCTCCCCTGCTGGGGCCCCTTCTCCTTTTGATA
AGGAAGCTGAAGGACAGTTTTATAACTATTTTCCCAACGGCAGCG
GCGCCACAAACTTCTCTCTGCTAAAGCAAGCAGGTGATGTTGAAG
AAAACCCCGGGCCTATGAGCTACTTTGTGGATTCTGCTGGGAGCA
GCCCCGTCCCTTACTCAGCGCCTCGTCCTGCAGTGGTGAGGCAAG
GACCTAGCAACACTTATGAAGATCCTCGAATGAACTGTGGTTTCC
AGTCCAATTATCACCAGCAAAGACCTTGCTACCCCTTTTGGGATG
AGATGGCAACTCAGGAAGTTCCTACTGGTCTTGAACACTGTGTCT
CAGATATGGAATGTGCAGATGTCCCACTATTAACTCCAAGCAGCA
AAGAAATGATGTCTCAAGCATTAAAAGCTACTTTCAGTGGTTTCA
CTAAAGAACAGCAACGACTGGGGATCCCAAAAGACCCCCGGCAGT
GGACAGAAACCCATGTTCGGGACTGGGTGATGTGGGCTGTGAATG
AATTCAGCCTGAAAGGTGTAGACTTCCAGAAGTTCTGTATGAATG
GAGCAGCCCTCTGCGCCCTGGGTAAAGACTGCTTTCTCGAGCTGG
CCCCAGACTTTGTTGGGGACATCTTATGGGAACATCTAGAGATCC
TGCAGAAAGAGGATGTGAAACCATATCAAGTTAATGGAGTCAACC
CAGCCTATCCAGAATCCCGCTATACCTCGGATTACTTCATTAGCT
ATGGTATTGAGCATGCCCAGTGTGTTCCACCATCGGAGTTCTCAG
AGCCCAGCTTCATCACAGAGTCCTATCAGACGCTCCATCCCATCA
GCTCGGAAGAGCTCCTCTCCCTCAAGTATGAGAATGACTACCCCT
CGGTCATTCTCCGAGACCCTCTCCAGACAGACACCTTGCAGAATG
ACTACTTTGCTATCAAACAAGAAGTCGTCACCCCAGACAACATGT
GCATGGGGAGGACCAGTCGTGGTAAACTCGGGGGCCAGGACTCTT
TTGAAAGCATAGAGAGCTACGATAGTTGTGATCGCCTCACCCAGT
CCTGGAGCAGCCAGTCATCTTTCAACAGCCTGCAGCGTGTTCCCT
CCTATGACAGCTTCGACTCAGAGGACTATCCGGCTGCCCTGCCCA
ACCACAAGCCCAAGGGCACCTTCAAGGACTATGTGCGGGACCGTG
CTGACCTCAATAAGGACAAGCCTGTCATTCCTGCTGCTGCCCTAG
CTGGCTACACAGGCAGTGGACCAATCCAGCTATGGCAGTTTCTTC
TGGAATTACTCACTGATAAATCCTGTCAGTCTTTTATCAGCTGGA
CAGGAGATGGCTGGGAATTCAAACTTTCTGACCCAGATGAGGTGG
CCAGGAGATGGGGAAAGAGGAAAAACAAACCTAAGATGAATTATG
AGAAACTGAGCCGTGGCCTACGCTACTATTACGACAAAAACATCA
TCCACAAGACAGCGGGGAAACGCTACGTGTACCGCTTTGTGTGTG
ACCTGCAGAGCCTGCTGGGGTACACCCCTGAGGAGCTGCACGCCA
TGCTGGACGTCAAGCCAGATGCCGACGAGGGCTCCGGCGAGGGCA
GGGGAAGTCTTCTAACATGCGGGGACGTGGAGGAAAATCCCGGCC
CAATGCCGCAGCTGGACTCCGGCGGGGGCGGCGCGGGCGGCGGCG
ACGACCTCGGCGCGCCGGACGAGCTGCTGGCCTTCCAGGATGAAG
GCGAGGAGCAGGACGACAAGAGCCGCGACAGCGCCGCCGGTCCCG
AGCGCGACCTGGCCGAGCTCAAGTCGTCGCTCGTGAACGAGTCCG
AGGGCGCGGCCGGCGGCGCAGGGATCCCGGGGGTCCCGGGGGCCG
GCGCCGGGGCCCGCGGCGAGGCCGAGGCTCTCGGGCGGGAACACG
CTGCGCAGAGACTCTTCCCGGACAAACTTCCAGAGCCCCTGGAGG
ACGGCCTGAAGGCCCCGGAGTGCACCAGCGGCATGTACAAAGAGA
CCGTCTACTCCGCCTTCAATCTGCTCATGCATTACCCACCCCCCT
CGGGAGCAGGGCAGCACCCCCAGCCGCAGCCCCCGCTGCACAAGG
CCAATCAGCCCCCCCACGGTGTCCCCCAACTCTCTCTCTACGAAC
ATTTCAACAGCCCACATCCCACCCCTGCACCTGCGGACATCAGCC
AGAAGCAAGTTCACAGGCCTCTGCAGACCCCTGACCTCTCTGGCT
TCTACTCCCTGACCTCAGGCAGCATGGGGCAGCTCCCCCACACTG
TGAGCTGGTTCACCCACCCATCCTTGATGCTAGGTTCTGGTGTAC
CTGGTCACCCAGCAGCCATCCCCCACCCGGCCATTGTGCCCCCCT
CAGGGAAGCAGGAGCTGCAGCCCTTCGACCGCAACCTGAAGACAC
AAGCAGAGTCCAAGGCAGAGAAGGAGGCCAAGAAGCCAACCATCA
AGAAGCCCCTCAATGCCTTCATGCTGTACATGAAGGAGATGAGAG
CCAAGGTCATTGCAGAGTGCACACTTAAGGAGAGCGCTGCCATCA
ACCAGATCCTGGGCCGCAGGTGGCACGCGCTGTCGCGAGAAGAGC
AGGCCAAGTACTATGAGCTGGCCCGCAAGGAGAGGCAGCTGCACA
TGCAGCTATACCCAGGCTGGTCAGCGCGGGACAACTACGGGAAGA
AGAAGAGGCGGTCGAGGGAAAAGCACCAAGAATCCACCACAGGAG
GAAAAAGAAATGCATTCGGTACTTACCCGGAGAAGGCCGCTGCCC
CAGCCCCGTTCCTTCCGATGACAGTGCTCTAG
m RNA exemplary mRNA encoding TBET-ETS1-TCF7
SEQ ID NO: 7
AUGGGCAUCGUGGAGCCGGGUUGCGGAGACAUGCUGACGGGCACC
GAGCCGAUGCCGGGGAGCGACGAGGGCCGGGCGCCUGGCGCCGAC
CCGCAGCACCGCUACUUCUACCCGGAGCCGGGCGCGCAGGACGCG
GACGAGCGUCGCGGGGGCGGCAGCCUGGGGUCUCCCUACCCGGGG
GGCGCCUUGGUGCCCGCCCCGCCGAGCCGCUUCCUUGGAGCCUAC
GCCUACCCGCCGCGACCCCAGGCGGCCGGCUUCCCCGGCGCGGGC
GAGUCCUUCCCGCCGCCCGCGGACGCCGAGGGCUACCAGCCGGGC
GAGGGCUACGCCGCCCCGGACCCGCGCGCCGGGCUCUACCCGGGG
CCGCGUGAGGACUACGCGCUACCCGCGGGACUGGAGGUGUCGGGG
AAACUGAGGGUCGCGCUCAACAACCACCUGUUGUGGUCCAAGUUU
AAUCAGCACCAGACAGAGAUGAUCAUCACCAAGCAGGGACGGCGG
AUGUUCCCAUUCCUGUCAUUUACUGUGGCCGGGCUGGAGCCCACC
AGCCACUACAGGAUGUUUGUGGACGUGGUCUUGGUGGACCAGCAC
CACUGGCGGUACCAGAGCGGCAAGUGGGUGCAGUGUGGAAAGGCC
GAGGGCAGCAUGCCAGGAAACCGCCUGUACGUCCACCCGGACUCC
CCCAACACAGGAGCGCACUGGAUGCGCCAGGAAGUUUCAUUUGGG
AAACUAAAGCUCACAAACAACAAGGGGGCGUCCAACAAUGUGACC
CAGAUGAUUGUGCUCCAGUCCCUCCAUAAGUACCAGCCCCGGCUG
CAUAUCGUUGAGGUGAACGACGGAGAGCCAGAGGCAGCCUGCAAC
GCUUCCAACACGCAUAUCUUUACUUUCCAAGAAACCCAGUUCAUU
GCCGUGACUGCCUACCAGAAUGCCGAGAUUACUCAGCUGAAAAUU
GAUAAUAACCCCUUUGCCAAAGGAUUCCGGGAGAACUUUGAGUCC
AUGUACACAUCUGUUGACACCAGCAUCCCCUCCCCGCCUGGACCC
AACUGUCAAUUCCUUGGGGGAGAUCACUACUCUCCUCUCCUACCC
AACCAGUAUCCUGUUCCCAGCCGCUUCUACCCCGACCUUCCUGGC
CAGGCGAAGGAUGUGGUUCCCCAGGCUUACUGGCUGGGGGCCCCC
CGGGACCACAGCUAUGAGGCUGAGUUUCGAGCAGUCAGCAUGAAG
CCUGCAUUCUUGCCCUCUGCCCCUGGGCCCACCAUGUCCUACUAC
CGAGGCCAGGAGGUCCUGGCACCUGGAGCUGGCUGGCCUGUGGCA
CCCCAGUACCCUCCCAAGAUGGGCCCGGCCAGCUGGUUCCGCCCU
AUGCGGACUCUGCCCAUGGAACCCGGCCCUGGAGGCUCAGAGGGA
CGGGGACCAGAGGACCAGGGUCCCCCCUUGGUGUGGACUGAGAUU
GCCCCCAUCCGGCCGGAAUCCAGUGAUUCAGGACUGGGCGAAGGA
GACUCUAAGAGGAGGCGCGUGUCCCCCUAUCCUUCCAGUGGUGAC
AGCUCCUCCCCUGCUGGGGCCCCUUCUCCUUUUGAUAAGGAAGCU
GAAGGACAGUUUUAUAACUAUUUUCCCAACGGCAGCGGCGCCACA
AACUUCUCUCUGCUAAAGCAAGCAGGUGAUGUUGAAGAAAACCCC
GGGCCUAUGAGCUACUUUGUGGAUUCUGCUGGGAGCAGCCCCGUC
CCUUACUCAGCGCCUCGUCCUGCAGUGGUGAGGCAAGGACCUAGC
AACACUUAUGAAGAUCCUCGAAUGAACUGUGGUUUCCAGUCCAAU
UAUCACCAGCAAAGACCUUGCUACCCCUUUUGGGAUGAGAUGGCA
ACUCAGGAAGUUCCUACUGGUCUUGAACACUGUGUCUCAGAUAUG
GAAUGUGCAGAUGUCCCACUAUUAACUCCAAGCAGCAAAGAAAUG
AUGUCUCAAGCAUUAAAAGCUACUUUCAGUGGUUUCACUAAAGAA
CAGCAACGACUGGGGAUCCCAAAAGACCCCCGGCAGUGGACAGAA
ACCCAUGUUCGGGACUGGGUGAUGUGGGCUGUGAAUGAAUUCAGC
CUGAAAGGUGUAGACUUCCAGAAGUUCUGUAUGAAUGGAGCAGCC
CUCUGCGCCCUGGGUAAAGACUGCUUUCUCGAGCUGGCCCCAGAC
UUUGUUGGGGACAUCUUAUGGGAACAUCUAGAGAUCCUGCAGAAA
GAGGAUGUGAAACCAUAUCAAGUUAAUGGAGUCAACCCAGCCUAU
CCAGAAUCCCGCUAUACCUCGGAUUACUUCAUUAGCUAUGGUAUU
GAGCAUGCCCAGUGUGUUCCACCAUCGGAGUUCUCAGAGCCCAGC
UUCAUCACAGAGUCCUAUCAGACGCUCCAUCCCAUCAGCUCGGAA
GAGCUCCUCUCCCUCAAGUAUGAGAAUGACUACCCCUCGGUCAUU
CUCCGAGACCCUCUCCAGACAGACACCUUGCAGAAUGACUACUUU
GCUAUCAAACAAGAAGUCGUCACCCCAGACAACAUGUGCAUGGGG
AGGACCAGUCGUGGUAAACUCGGGGGCCAGGACUCUUUUGAAAGC
AUAGAGAGCUACGAUAGUUGUGAUCGCCUCACCCAGUCCUGGAGC
AGCCAGUCAUCUUUCAACAGCCUGCAGCGUGUUCCCUCCUAUGAC
AGCUUCGACUCAGAGGACUAUCCGGCUGCCCUGCCCAACCACAAG
CCCAAGGGCACCUUCAAGGACUAUGUGCGGGACCGUGCUGACCUC
AAUAAGGACAAGCCUGUCAUUCCUGCUGCUGCCCUAGCUGGCUAC
ACAGGCAGUGGACCAAUCCAGCUAUGGCAGUUUCUUCUGGAAUUA
CUCACUGAUAAAUCCUGUCAGUCUUUUAUCAGCUGGACAGGAGAU
GGCUGGGAAUUCAAACUUUCUGACCCAGAUGAGGUGGCCAGGAGA
UGGGGAAAGAGGAAAAACAAACCUAAGAUGAAUUAUGAGAAACUG
AGCCGUGGCCUACGCUACUAUUACGACAAAAACAUCAUCCACAAG
ACAGCGGGGAAACGCUACGUGUACCGCUUUGUGUGUGACCUGCAG
AGCCUGCUGGGGUACACCCCUGAGGAGCUGCACGCCAUGCUGGAC
GUCAAGCCAGAUGCCGACGAGGGCUCCGGCGAGGGCAGGGGAAGU
CUUCUAACAUGCGGGGACGUGGAGGAAAAUCCCGGCCCAAUGCCG
CAGCUGGACUCCGGCGGGGGCGGCGCGGGCGGCGGCGACGACCUC
GGCGCGCCGGACGAGCUGCUGGCCUUCCAGGAUGAAGGCGAGGAG
CAGGACGACAAGAGCCGCGACAGCGCCGCCGGUCCCGAGCGCGAC
CUGGCCGAGCUCAAGUCGUCGCUCGUGAACGAGUCCGAGGGCGCG
GCCGGCGGCGCAGGGAUCCCGGGGGUCCCGGGGGCCGGCGCCGGG
GCCCGCGGCGAGGCCGAGGCUCUCGGGCGGGAACACGCUGCGCAG
AGACUCUUCCCGGACAAACUUCCAGAGCCCCUGGAGGACGGCCUG
AAGGCCCCGGAGUGCACCAGCGGCAUGUACAAAGAGACCGUCUAC
UCCGCCUUCAAUCUGCUCAUGCAUUACCCACCCCCCUCGGGAGCA
GGGCAGCACCCCCAGCCGCAGCCCCCGCUGCACAAGGCCAAUCAG
CCCCCCCACGGUGUCCCCCAACUCUCUCUCUACGAACAUUUCAAC
AGCCCACAUCCCACCCCUGCACCUGCGGACAUCAGCCAGAAGCAA
GUUCACAGGCCUCUGCAGACCCCUGACCUCUCUGGCUUCUACUCC
CUGACCUCAGGCAGCAUGGGGCAGCUCCCCCACACUGUGAGCUGG
UUCACCCACCCAUCCUUGAUGCUAGGUUCUGGUGUACCUGGUCAC
CCAGCAGCCAUCCCCCACCCGGCCAUUGUGCCCCCCUCAGGGAAG
CAGGAGCUGCAGCCCUUCGACCGCAACCUGAAGACACAAGCAGAG
UCCAAGGCAGAGAAGGAGGCCAAGAAGCCAACCAUCAAGAAGCCC
CUCAAUGCCUUCAUGCUGUACAUGAAGGAGAUGAGAGCCAAGGUC
AUUGCAGAGUGCACACUUAAGGAGAGCGCUGCCAUCAACCAGAUC
CUGGGCCGCAGGUGGCACGCGCUGUCGCGAGAAGAGCAGGCCAAG
UACUAUGAGCUGGCCCGCAAGGAGAGGCAGCUGCACAUGCAGCUA
UACCCAGGCUGGUCAGCGCGGGACAACUACGGGAAGAAGAAGAGG
CGGUCGAGGGAAAAGCACCAAGAAUCCACCACAGGAGGAAAAAGA
AAUGCAUUCGGUACUUACCCGGAGAAGGCCGCUGCCCCAGCCCCG
UUCCUUCCGAUGACAGUGCUCUAG

1. A method comprising
(a) providing a vertebrate somatic cell,
(b) inducing expression in said cell of at least three transcription factors, wherein said transcription factors are:
Tbet (Tbx21),
Tcf7, and
Ets1;
(c) incubating said cell to allow transdifferentiation.

2-12. (canceled)

13. A method according to claim 1 wherein said cell is transdifferentiated to an immune effector cell.

14. A method according to claim 1 wherein said cell is transdifferentiated to an alpha/beta T cell, or a gamma/delta T cell.

15. A method according to claim 1 wherein said cell is transdifferentiated to a CD8+ T cell.

16. A method according to claim 1 wherein said cell is transdifferentiated to a CD4+ T cell.

17. A method according to claim 1 wherein the vertebrate somatic cell is selected from the group consisting of: a mesenchymal stem cell (MSC), a skin fibroblast, an endothelial cell, a keratinocyte, and a hepatocyte.

18. (canceled)

19. A method according to claim 17 wherein the vertebrate somatic cell is a mesenchymal stem cell.

20. A method according to claim 17 wherein the vertebrate somatic cell is a skin fibroblast.

21. A method according to claim 17 wherein the vertebrate somatic cell is a human mesenchymal stem cell (hMSC), and wherein said hMSC is derived from bone marrow.

22. A method according to claim 1 wherein the immune effector cell of (c) is a cell which expresses one or more gene(s) selected from the group consisting of: CD3, CD45, CD2, CD5, CD7, CD4, and CD8.

23. A method according to claim 1 wherein the immune effector cell of (c) is a cell which expresses one or more gene(s) selected from the list consisting of: CD105 and CD73 at a lower level compared to the expression of the same gene(s) in a vertebrate somatic cell of (a).

24. A method according to claim 1 wherein the immune effector cell of (c) is a cell which does not express one or more gene(s) selected from the list consisting of: CD105 and CD73.

25. (canceled)

26. A method according to claim 1 wherein inducing expression of at least three transcription factors comprises:
providing nucleic acid(s) comprising three nucleotide sequences, each nucleotide sequence encoding a transcription factor selected from said at least three transcription factors, each nucleotide sequence being operatively linked to a promoter sequence capable of directing expression of said transcription factor; and
introducing said nucleic acid into said cell.

27. A method according to claim 26, wherein introducing said nucleic acid into said cell comprises electroporation of said cell.

28. A cell obtained by the method according to claim 1.

29. An isolated nucleic acid comprising nucleotide sequences encoding Tbet, Ets1 and Tcf7, each nucleotide sequence being operatively linked to a promoter sequence capable of directing expression of said nucleotide sequence encoding Tbet, Ets1 and Tcf7.

30. An isolated nucleic acid according to claim 29, wherein each said nucleotide sequence encoding Tbet, Ets1 and Tcf7 is operatively linked to a respective promoter sequence.

31. An isolated nucleic acid according to claim 29, wherein said nucleotide sequences encoding Tbet, Ets1 and Tcf7 are all operatively linked to a single promoter sequence.

32. An isolated nucleic acid according to claim 29, wherein said nucleotide sequences are arranged in the order 5′-promoter sequence-nucleotide sequence encoding Tbet-nucleotide sequence encoding Ets1-nucleotide sequence encoding Tcf7-3′.

33-35. (canceled)

36. A method of treating a subject comprising administering to said subject a cell according to claim 28.
